SEQUENCE LISTING (196 sequences)

SEQ ID NO: 1 - DNA, 321 bp, Mus musculus
  CDS (1)..(321)
gacattcagatgacccagtctcctgcctcccagtctgcatctctgggagaaagtgtcaccatcacatgcctggcaagtcagaccattgatacatggttagcatggtatcagcagaaaccagggaaatctcctcagctcctgatttatgctgccaccaacttggcagatggggtcccatcaaggttcagtggcagtggatctggcacaaaattttctttcaagatcagcagcctacaggctgaagattttgtaaattattactgtcaacaagtttacagttctccattcacgttcggtgctgggaccaagctggagctgaaa

SEQ ID NO: 2 - protein, 107 aa, Mus musculus
DIQMTQSPASQSASLGESVTITCLASQTIDTWLAWYQQKPGKSPQLLIYAATNLADGVPSRFSGSGSGTKFSFKISSLQAEDFVNYYCQQVYSSPFTFGAGTKLELK

SEQ ID NO: 3 - DNA, 351 bp, Mus musculus
  CDS (1)..(351)
gagatccagctgcagcagtctggacctgagctggtgaagcctggggcttcagtgcaggtatcctgcaagacttctggttactcattcactgactacaacgtgtactgggtgaggcagagccatggaaagagccttgagtggattggatatattgatccttacaatggtattactatctacgaccagaacttcaagggcaaggccacattgactgttgacaagtcttccaccacagccttcatgcatctcaacagcctgacatctgacgactctgcagtttatttctgtgcaagagatgtgactacggcccttgacttctggggccaaggcaccactctcacagtctcctca

SEQ ID NO: 4 - protein, 117 aa, Mus musculus
EIQLQQSGPELVKPGASVQVSCKTSGYSFTDYNVYWVRQSHGKSLEWIGYIDPYNGITIYDQNFKGKATLTVDKSSTTAFMHLNSLTSDDSAVYFCARDVTTALDFWGQGTTLTVSS

SEQ ID NO: 5 - protein, 7 aa, Mus musculus
LASQTID

SEQ ID NO: 6 - protein, 7 aa, Mus musculus
AATNLAD

SEQ ID NO: 7 - protein, 9 aa, Mus musculus
QQVYSSPFT

SEQ ID NO: 8 - protein, 6 aa, Mus musculus
TDYNVY

SEQ ID NO: 9 - protein, 17 aa, Mus musculus
YIDPYNGITIYDQNFKG

SEQ ID NO: 10 - protein, 8 aa, Mus musculus
DVTTALDF

SEQ ID NO: 11 - DNA, 21 bp, Mus musculus
ctggcaagtcagaccattgat

SEQ ID NO: 12 - DNA, 21 bp, Mus musculus
gctgccaccaacttggcagat

SEQ ID NO: 13 - DNA, 28 bp, Mus musculus
caacaagtttacagttctccattcacgt

SEQ ID NO: 14 - DNA, 18 bp, Mus musculus
actgactacaacgtgtac

SEQ ID NO: 15 - DNA, 51 bp, Mus musculus
tatattgatccttacaatggtattactatctacgaccagaacttcaagggc

SEQ ID NO: 16 - DNA, 24 bp, Mus musculus
gatgtgactacggcccttgacttc

SEQ ID NO: 17 - DNA, 23 bp, Artificial Sequence
  Description of Artificial Sequence Synthetic oligonucleotide
gcacctccagatgttaactgctc

SEQ ID NO: 18 - DNA, 20 bp, Artificial Sequence
  Description of Artificial Sequence Synthetic oligonucleotide
gaartavcccttgaccaggc

SEQ ID NO: 19 - DNA, 35 bp, Artificial Sequence
  Description of Artificial Sequence Synthetic oligonucleotide
ggaggcggcggttctgacattgtgmtgwcmcartc

SEQ ID NO: 20 - DNA, 45 bp, Artificial Sequence
  Description of Artificial Sequence Synthetic oligonucleotide
atttcaggcccagccggccatggccgargtycarctkcarcaryc

SEQ ID NO: 21 - DNA, 33 bp, Artificial Sequence
  Description of Artificial Sequence Synthetic oligonucleotide
cccgggccaccatgkccccwrctcagytyctkg

SEQ ID NO: 22 - DNA, 35 bp, Artificial Sequence
  Description of Artificial Sequence Synthetic oligonucleotide
cccgggccaccatggratgsagctgkgtmatsctc

SEQ ID NO: 23 - DNA, 52 bp, Artificial Sequence
  Description of Artificial Sequence Synthetic oligonucleotide
atatactcgcgacagctacaggtgtccactccgagatccagctgcagcagtc

SEQ ID NO: 24 - DNA, 31 bp, Artificial Sequence
  Description of Artificial Sequence Synthetic oligonucleotide
gacctgaattctaaggagactgtgagagtgg

SEQ ID NO: 25 - DNA, 29 bp, Artificial Sequence
  Description of Artificial Sequence Synthetic oligonucleotide
ttaattgatatccagatgacccagtctcc

SEQ ID NO: 26 - DNA, 45 bp, Artificial Sequence
  Description of Artificial Sequence Synthetic oligonucleotide
taatcgttcgaaaagtgtacttacgtttcagctccagcttggtcc

SEQ ID NO: 27 - protein, 23 aa, Mus musculus
DIQMTQSPASQSASLGESVTITC

SEQ ID NO: 28 - protein, 15 aa, Mus musculus
WYQQKPGKSPQLLIY

SEQ ID NO: 29 - protein, 23 aa, Homo sapiens
DIQMTQSPASLSASVGDRVTITC

SEQ ID NO: 30 - protein, 15 aa, Homo sapiens
WYLQKPGKSPQLLIY

SEQ ID NO: 31 - protein, 32 aa, Mus musculus
GVPSRFSGSGSGTKFSFKISSLQAEDFVNYYC

SEQ ID NO: 32 - protein, 10 aa, Mus musculus
FGAGTKLELK

SEQ ID NO: 33 - protein, 32 aa, Homo sapiens
GVPSRFSGSGSGTDFSFTISSLQPEDFATYYC

SEQ ID NO: 34 - protein, 10 aa, Homo sapiens
FGQGTKLEIK

SEQ ID NO: 35 - protein, 30 aa, Mus musculus
EIQLQQSGPELVKPGASVQVSCKTSGYSFT

SEQ ID NO: 36 - protein, 30 aa, Homo sapiens
QIQLVQSGGEVKKPGASVRVSCKASGYSFT

SEQ ID NO: 37 - protein, 32 aa, Mus musculus
KATLTVDKSSTTAFMHLNSLTSDDSAVYFCAR

SEQ ID NO: 38 - protein, 11 aa, Mus musculus
WGQGTTLTVSS

SEQ ID NO: 39 - protein, 32 aa, Homo sapiens
KATLTVDKSTSTAYMELSSLRSEDTAVYFCAR

SEQ ID NO: 40 - protein, 11 aa, Homo sapiens
WGQGTTVTVSS

SEQ ID NO: 41 - DNA, 37 bp, Artificial Sequence
  Description of Artificial Sequence Synthetic primer
tttcgtacgtcttgtcccagatccagctgcagcagtc

SEQ ID NO: 42 - DNA, 43 bp, Artificial Sequence
  Description of Artificial Sequence Synthetic primer
agcgaattctgaggagactgtgacagtggtgccttggccccag

SEQ ID NO: 43 - DNA, 38 bp, Artificial Sequence
  Description of Artificial Sequence Synthetic primer
gtgaggcagagccctggaaagggccttgagtggattgg

SEQ ID NO: 44 - DNA, 38 bp, Artificial Sequence
  Description of Artificial Sequence Synthetic primer
ccaatccactcaaggccctttccagggctctgcctcac

SEQ ID NO: 45 - DNA, 47 bp, Artificial Sequence
  Description of Artificial Sequence Synthetic primer
gcatctcaacagcctgagatctgaagacactgcagtttatttctgtg

SEQ ID NO: 46 - DNA, 43 bp, Artificial Sequence
  Description of Artificial Sequence Synthetic primer
ctgcagtgtcttcagatctcaggctgttgagatgcatgaaggc

SEQ ID NO: 47 - DNA, 43 bp, Artificial Sequence
  Description of Artificial Sequence Synthetic primer
gtcttcagatctcaggctgctgagctccatgaaggctgtggtg

SEQ ID NO: 48 - DNA, 25 bp, Artificial Sequence
  Description of Artificial Sequence Synthetic primer
tacgactcactatagggcgaattgg

SEQ ID NO: 49 - DNA, 43 bp, Artificial Sequence
  Description of Artificial Sequence Synthetic primer
ctgttgacaagtctaccagcacagcctacatggagctcagcag

SEQ ID NO: 50 - DNA, 43 bp, Artificial Sequence
  Description of Artificial Sequence Synthetic primer
ctgctgagctccatgtaggctgtgctggtagacttgtcaacag

SEQ ID NO: 51 - DNA, 47 bp, Artificial Sequence
  Description of Artificial Sequence Synthetic primer
gcactgaagccccaggcttcaccagctcacctccagactgctgcagc

SEQ ID NO: 52 - DNA, 49 bp, Artificial Sequence
  Description of Artificial Sequence Synthetic primer
ctggggcttcagtgcgggtatcctgcaaggcttctggttactcattcac

SEQ ID NO: 53 - DNA, 46 bp, Artificial Sequence
  Description of Artificial Sequence Synthetic primer
tcgtacgtcttgtcccagatccagctggtgcagtctggaggtgagc

SEQ ID NO: 54 - DNA, 44 bp, Artificial Sequence
  Description of Artificial Sequence Synthetic primer
gcactgaagccccaggcttcttcacctcacctccagactgcacc

SEQ ID NO: 55 - DNA, 32 bp, Artificial Sequence
  Description of Artificial Sequence Synthetic primer
gcagtctggacctgagctgaagaagcctgggg

SEQ ID NO: 56 - DNA, 32 bp, Artificial Sequence
  Description of Artificial Sequence Synthetic primer
ccccaggcttcttcagctcaggtccagactgc

SEQ ID NO: 57 - DNA, 33 bp, Artificial Sequence
  Description of Artificial Sequence Synthetic primer
gctggtgcagtctggacctgaggtgaagaagcc

SEQ ID NO: 58 - DNA, 33 bp, Artificial Sequence
  Description of Artificial Sequence Synthetic primer
ggcttcttcacctcaggtccagactgcaccagc

SEQ ID NO: 59 - DNA, 36 bp, Artificial Sequence
  Description of Artificial Sequence Synthetic primer
gcagtctggacctgagctggtgaagcctggggcttc

SEQ ID NO: 60 - DNA, 36 bp, Artificial Sequence
  Description of Artificial Sequence Synthetic primer
gaagccccaggcttcaccagctcaggtccagactgc

SEQ ID NO: 61 - DNA, 30 bp, Artificial Sequence
  Description of Artificial Sequence Synthetic primer
cagtctggacctgaggtggtgaagcctggg

SEQ ID NO: 62 - DNA, 30 bp, Artificial Sequence
  Description of Artificial Sequence Synthetic primer
cccaggcttcaccacctcaggtccagactg

SEQ ID NO: 63 - DNA, 42 bp, Artificial Sequence
  Description of Artificial Sequence Synthetic primer
ttcgaaaagtgtacttacgtttgatctccagcttggtcccag

SEQ ID NO: 64 - DNA, 29 bp, Artificial Sequence
  Description of Artificial Sequence Synthetic primer
accggtgatatccagatgacccagtctcc

SEQ ID NO: 65 - DNA, 29 bp, Artificial Sequence
  Description of Artificial Sequence Synthetic primer
ggttagcatggtatctgcagaaaccaggg

SEQ ID NO: 66 - DNA, 29 bp, Artificial Sequence
  Description of Artificial Sequence Synthetic primer
ccctggtttctgcagataccatgctaacc

SEQ ID NO: 67 - DNA, 25 bp, Artificial Sequence
  Description of Artificial Sequence Synthetic primer
tacgactcactatagggcgaattgg

SEQ ID NO: 68 - DNA, 31 bp, Artificial Sequence
  Description of Artificial Sequence Synthetic primer
ccacagatgcagacagggaggcaggagactg

SEQ ID NO: 69 - DNA, 51 bp, Artificial Sequence
  Description of Artificial Sequence Synthetic primer
ttcgaaaagtgtacttacgtttgatctccagcttggtaccagcaccgaacg

SEQ ID NO: 70 - DNA, 40 bp, Artificial Sequence
  Description of Artificial Sequence Synthetic primer
cctgtctgcatctgtgggagatagggtcaccatcacatgc

SEQ ID NO: 71 - DNA, 36 bp, Artificial Sequence
  Description of Artificial Sequence Synthetic primer
gatctccagcttggtaccctgaccgaacgtgaatgg

SEQ ID NO: 72 - DNA, 40 bp, Artificial Sequence
  Description of Artificial Sequence Synthetic primer
gtaggctgctgatcgtgaaagaaaagtctgtgccagatcc

SEQ ID NO: 73 - DNA, 47 bp, Artificial Sequence
  Description of Artificial Sequence Synthetic primer
cacgatcagcagcctacagcctgaagattttgtaaattattactgtc

SEQ ID NO: 74 - DNA, 45 bp, Artificial Sequence
  Description of Artificial Sequence Synthetic primer
gcagcctacagcctgaagattttgcaacttattactgtcaacaag

SEQ ID NO: 75 - DNA, 45 bp, Artificial Sequence
  Description of Artificial Sequence Synthetic primer
cttgttgacagtaataagttgcaaaatcttcaggctgtaggctgc

SEQ ID NO: 76 - DNA, 44 bp, Artificial Sequence
  Description of Artificial Sequence Synthetic primer
cagcagcctacagcctgaagattttgcaaattattactgtcaac

SEQ ID NO: 77 - DNA, 44 bp, Artificial Sequence
  Description of Artificial Sequence Synthetic primer
gttgacagtaataatttgcaaaatcttcaggctgtaggctgctg

SEQ ID NO: 78 - DNA, 40 bp, Artificial Sequence
  Description of Artificial Sequence Synthetic primer
cagtggatctggcacaaagttttctttcacgatcagcagc

SEQ ID NO: 79 - DNA, 40 bp, Artificial Sequence
  Description of Artificial Sequence Synthetic primer
gctgctgatcgtgaaagaaaactttgtgccagatccactg

SEQ ID NO: 80 - DNA, 33 bp, Artificial Sequence
  Description of Artificial Sequence Synthetic primer
ctgcagaaaccagggcaatctcctcagctcctg

SEQ ID NO: 81 - DNA, 33 bp, Artificial Sequence
  Description of Artificial Sequence Synthetic primer
caggagctgaggagattgccctggtttctgcag

SEQ ID NO: 82 - protein, 23 aa, Mus musculus
DIQMTQSPASQSASLGESVTITC

SEQ ID NO: 83 - protein, 15 aa, Mus musculus
WYQQKPGKSPQLLIY

SEQ ID NO: 84 - protein, 32 aa, Mus musculus; identical to SEQ ID NO: 31

SEQ ID NO: 85 - protein, 10 aa, Mus musculus
FGAGTKLELK

SEQ ID NO: 86 - protein, 23 aa, Artificial Sequence
  Description of Artificial Sequence Synthetic peptide
DIQMTQSPASQSASLGESVTITC

SEQ ID NO: 87 - protein, 15 aa, Artificial Sequence
  Description of Artificial Sequence Synthetic peptide
WYQQKPGKSPQLLIY

SEQ ID NO: 88 - protein, 32 aa, Artificial Sequence; identical to SEQ ID NO: 31
  Description of Artificial Sequence Synthetic peptide

SEQ ID NO: 89 - protein, 10 aa, Artificial Sequence
  Description of Artificial Sequence Synthetic peptide
FGAGTKLEIK

SEQ ID NO: 90 - protein, 23 aa, Artificial Sequence
  Description of Artificial Sequence Synthetic peptide
DIQMTQSPASQSASLGESVTITC

SEQ ID NO: 91 - protein, 15 aa, Artificial Sequence
  Description of Artificial Sequence Synthetic peptide
WYLQKPGKSPQLLIY

SEQ ID NO: 92 - protein, 31 aa, Artificial Sequence
  Description of Artificial Sequence Synthetic peptide
GVPSFSGSGSGTKFSFKISSLQAEDFVNYYC

SEQ ID NO: 93 - protein, 10 aa, Artificial Sequence
  Description of Artificial Sequence Synthetic peptide
FGAGTKLEIK

SEQ ID NO: 94 - protein, 23 aa, Artificial Sequence
  Description of Artificial Sequence Synthetic peptide
DIQMTQSPASLSASVGDRVTITC

SEQ ID NO: 95 - protein, 15 aa, Artificial Sequence
  Description of Artificial Sequence Synthetic peptide
WYLQKPGKSPQLLIY

SEQ ID NO: 96 - protein, 32 aa, Artificial Sequence; identical to SEQ ID NO: 31
  Description of Artificial Sequence Synthetic peptide

SEQ ID NO: 97 - protein, 10 aa, Artificial Sequence
  Description of Artificial Sequence Synthetic peptide
FGQGTKLEIK

SEQ ID NO: 98 - protein, 23 aa, Artificial Sequence
  Description of Artificial Sequence Synthetic peptide
DIQMTQSPASQSASLGESVTITC

SEQ ID NO: 99 - protein, 15 aa, Artificial Sequence
  Description of Artificial Sequence Synthetic peptide
WYLQKPGKSPQLLIY

SEQ ID NO: 100 - protein, 32 aa, Artificial Sequence; identical to SEQ ID NO: 31
  Description of Artificial Sequence Synthetic peptide

SEQ ID NO: 101 - protein, 10 aa, Artificial Sequence
  Description of Artificial Sequence Synthetic peptide
FGQGTKLEIK

SEQ ID NO: 102 - protein, 23 aa, Artificial Sequence
  Description of Artificial Sequence Synthetic peptide
DIQMTQSPASQSASLGESVTITC

SEQ ID NO: 103 - protein, 15 aa, Artificial Sequence
  Description of Artificial Sequence Synthetic peptide
WYLQKPGKSPQLLIY

SEQ ID NO: 104 - protein, 32 aa, Artificial Sequence
  Description of Artificial Sequence Synthetic peptide
GVPSRFSGSGSGTDFSFTISSLQPEDFVNYYC

SEQ ID NO: 105 - protein, 10 aa, Artificial Sequence
  Description of Artificial Sequence Synthetic peptide
FGQGTKLEIK

SEQ ID NO: 106 - protein, 23 aa, Artificial Sequence
  Description of Artificial Sequence Synthetic peptide
DIQMTQSPASQSASLGESVTITC

SEQ ID NO: 107 - protein, 15 aa, Artificial Sequence
  Description of Artificial Sequence Synthetic peptide
WYLQKPGKSPQLLIY

SEQ ID NO: 108 - protein, 32 aa, Artificial Sequence; identical to SEQ ID NO: 33
  Description of Artificial Sequence Synthetic peptide

SEQ ID NO: 109 - protein, 10 aa, Artificial Sequence
  Description of Artificial Sequence Synthetic peptide
FGQGTKLEIK

SEQ ID NO: 110 - protein, 23 aa, Artificial Sequence
  Description of Artificial Sequence Synthetic peptide
DIQMTQSPASLSASVGDRVTITC

SEQ ID NO: 111 - protein, 15 aa, Artificial Sequence
  Description of Artificial Sequence Synthetic peptide
WYLQKPGKSPQLLIY

SEQ ID NO: 112 - protein, 32 aa, Artificial Sequence; identical to SEQ ID NO: 33
  Description of Artificial Sequence Synthetic peptide

SEQ ID NO: 113 - protein, 10 aa, Artificial Sequence
  Description of Artificial Sequence Synthetic peptide
FGQGTKLEIK

SEQ ID NO: 114 - protein, 23 aa, Artificial Sequence
  Description of Artificial Sequence Synthetic peptide
DIQMTQSPASLSASVGDRVTITC

SEQ ID NO: 115 - protein, 15 aa, Artificial Sequence
  Description of Artificial Sequence Synthetic peptide
WYLQKPGKSPQLLIY

SEQ ID NO: 116 - protein, 32 aa, Artificial Sequence
  Description of Artificial Sequence Synthetic peptide
GVPSRFSGSGSGTDFSFTISSLQPEDFANYYC

SEQ ID NO: 117 - protein, 10 aa, Artificial Sequence
  Description of Artificial Sequence Synthetic peptide
FGQGTKLEIK

SEQ ID NO: 118 - protein, 23 aa, Artificial Sequence
  Description of Artificial Sequence Synthetic peptide
DIQMTQSPASLSASVGDRVTITC

SEQ ID NO: 119 - protein, 15 aa, Artificial Sequence
  Description of Artificial Sequence Synthetic peptide
WYLQKPGKSPQLLIY

SEQ ID NO: 120 - protein, 32 aa, Artificial Sequence
  Description of Artificial Sequence Synthetic peptide
GVPSRFSGSGSGTKFSFTISSLQPEDFANYYC

SEQ ID NO: 121 - protein, 10 aa, Artificial Sequence
  Description of Artificial Sequence Synthetic peptide
FGQGTKLEIK

SEQ ID NO: 122 - protein, 23 aa, Artificial Sequence
  Description of Artificial Sequence Synthetic peptide
DIQMTQSPASLSASVGDRVTITC

SEQ ID NO: 123 - protein, 15 aa, Artificial Sequence
  Description of Artificial Sequence Synthetic peptide
WYLQKPGQSPQLLIY

SEQ ID NO: 124 - protein, 32 aa, Artificial Sequence; identical to SEQ ID NO: 120
  Description of Artificial Sequence Synthetic peptide

SEQ ID NO: 125 - protein, 10 aa, Artificial Sequence
  Description of Artificial Sequence Synthetic peptide
FGQGTKLEIK

SEQ ID NO: 126 - protein, 11 aa, Mus musculus
LASQTIDTWLA

SEQ ID NO: 127 - protein, 7 aa, Mus musculus
AATNLAD

SEQ ID NO: 128 - protein, 9 aa, Mus musculus
QQVYSSPFT

SEQ ID NO: 129 - protein, 30 aa, Mus musculus; identical to SEQ ID NO: 35

SEQ ID NO: 130 - protein, 14 aa, Mus musculus
WVRQSHGKSLEWIG

SEQ ID NO: 131 - protein, 32 aa, Mus musculus; identical to SEQ ID NO: 37

SEQ ID NO: 132 - protein, 11 aa, Mus musculus
WGQGTTLTVSS

SEQ ID NO: 133 - protein, 30 aa, Artificial Sequence
  Description of Artificial Sequence Synthetic peptide
QIQLQQSGPELVKPGASVQVSCKTSGYSFT

SEQ ID NO: 134 - protein, 14 aa, Artificial Sequence
  Description of Artificial Sequence Synthetic peptide
WVRQSHGKSLEWIG

SEQ ID NO: 135 - protein, 32 aa, Artificial Sequence; identical to SEQ ID NO: 37
  Description of Artificial Sequence Synthetic peptide

SEQ ID NO: 136 - protein, 11 aa, Artificial Sequence
  Description of Artificial Sequence Synthetic peptide
WGQGTTVTVSS

SEQ ID NO: 137 - protein, 30 aa, Artificial Sequence; identical to SEQ ID NO: 133
  Description of Artificial Sequence Synthetic peptide

SEQ ID NO: 138 - protein, 14 aa, Artificial Sequence
  Description of Artificial Sequence Synthetic peptide
WVRQSPGKGLEWIG

SEQ ID NO: 139 - protein, 32 aa, Artificial Sequence; identical to SEQ ID NO: 37
  Description of Artificial Sequence Synthetic peptide

SEQ ID NO: 140 - protein, 11 aa, Artificial Sequence
  Description of Artificial Sequence Synthetic peptide
WGQGTTVTVSS

SEQ ID NO: 141 - protein, 30 aa, Artificial Sequence; identical to SEQ ID NO: 133
  Description of Artificial Sequence Synthetic peptide

SEQ ID NO: 142 - protein, 14 aa, Artificial Sequence
  Description of Artificial Sequence Synthetic peptide
WVRQSPGKGLEWIG

SEQ ID NO: 143 - protein, 32 aa, Artificial Sequence
  Description of Artificial Sequence Synthetic peptide
KATLTVDKSSTTAFMHLNSLRSEDTAVYFCAR

SEQ ID NO: 144 - protein, 11 aa, Artificial Sequence
  Description of Artificial Sequence Synthetic peptide
WGQGTTVTVSS

SEQ ID NO: 145 - protein, 30 aa, Artificial Sequence; identical to SEQ ID NO: 133
  Description of Artificial Sequence Synthetic peptide

SEQ ID NO: 146 - protein, 14 aa, Artificial Sequence
  Description of Artificial Sequence Synthetic peptide
WVRQSPGKGLEWIG

SEQ ID NO: 147 - protein, 32 aa, Artificial Sequence
  Description of Artificial Sequence Synthetic peptide
KATLTVDKSSTTAFMELSSLRSEDTAVYFCAR

SEQ ID NO: 148 - protein, 11 aa, Artificial Sequence
  Description of Artificial Sequence Synthetic peptide
WGQGTTVTVSS

SEQ ID NO: 149 - protein, 30 aa, Artificial Sequence; identical to SEQ ID NO: 133
  Description of Artificial Sequence Synthetic peptide

SEQ ID NO: 150 - protein, 14 aa, Artificial Sequence
  Description of Artificial Sequence Synthetic peptide
WVRQSPGKGLEWIG

SEQ ID NO: 151 - protein, 32 aa, Artificial Sequence; identical to SEQ ID NO: 39
  Description of Artificial Sequence Synthetic peptide

SEQ ID NO: 152 - protein, 11 aa, Artificial Sequence
  Description of Artificial Sequence Synthetic peptide
WGQGTTVTVSS

SEQ ID NO: 153 - protein, 30 aa, Artificial Sequence
  Description of Artificial Sequence Synthetic peptide
QMQLQQSGGELVKPGASVRVSCKASGYSFT

SEQ ID NO: 154 - protein, 14 aa, Artificial Sequence
  Description of Artificial Sequence Synthetic peptide
WVRQSPGKGLEWIG

SEQ ID NO: 155 - protein, 32 aa, Artificial Sequence; identical to SEQ ID NO: 39
  Description of Artificial Sequence Synthetic peptide

SEQ ID NO: 156 - protein, 11 aa, Artificial Sequence
  Description of Artificial Sequence Synthetic peptide
WGQGTTVTVSS

SEQ ID NO: 157 - protein, 30 aa, Artificial Sequence
  Description of Artificial Sequence Synthetic peptide
QIQLVQSGGELVKPGASVRVSCKASGYSFT

SEQ ID NO: 158 - protein, 14 aa, Artificial Sequence
  Description of Artificial Sequence Synthetic peptide
WVRQSPGKGLEWIG

SEQ ID NO: 159 - protein, 32 aa, Artificial Sequence; identical to SEQ ID NO: 39
  Description of Artificial Sequence Synthetic peptide

SEQ ID NO: 160 - protein, 11 aa, Artificial Sequence
  Description of Artificial Sequence Synthetic peptide
WGQGTTVTVSS

SEQ ID NO: 161 - protein, 30 aa, Artificial Sequence; identical to SEQ ID NO: 36
  Description of Artificial Sequence Synthetic peptide

SEQ ID NO: 162 - protein, 14 aa, Artificial Sequence
  Description of Artificial Sequence Synthetic peptide
WVRQSPGKGLEWIG

SEQ ID NO: 163 - protein, 32 aa, Artificial Sequence; identical to SEQ ID NO: 39
  Description of Artificial Sequence Synthetic peptide

SEQ ID NO: 164 - protein, 11 aa, Artificial Sequence
  Description of Artificial Sequence Synthetic peptide
WGQGTTVTVSS

SEQ ID NO: 165 - protein, 30 aa, Artificial Sequence; identical to SEQ ID NO: 36
  Description of Artificial Sequence Synthetic peptide

SEQ ID NO: 166 - protein, 14 aa, Artificial Sequence
  Description of Artificial Sequence Synthetic peptide
WVRQSPGKGLEWIG

SEQ ID NO: 167 - protein, 32 aa, Artificial Sequence; identical to SEQ ID NO: 39
  Description of Artificial Sequence Synthetic peptide

SEQ ID NO: 168 - protein, 11 aa, Artificial Sequence
  Description of Artificial Sequence Synthetic peptide
WGQGTTVTVSS

SEQ ID NO: 169 - protein, 30 aa, Artificial Sequence
  Description of Artificial Sequence Synthetic peptide
QIQLVQSGPEVKKPGASVRVSCKASGYSFT

SEQ ID NO: 170 - protein, 14 aa, Artificial Sequence
  Description of Artificial Sequence Synthetic peptide
WVRQSPGKGLEWIG

SEQ ID NO: 171 - protein, 32 aa, Artificial Sequence; identical to SEQ ID NO: 39
  Description of Artificial Sequence Synthetic peptide

SEQ ID NO: 172 - protein, 11 aa, Artificial Sequence
  Description of Artificial Sequence Synthetic peptide
WGQGTTVTVSS

SEQ ID NO: 173 - protein, 30 aa, Artificial Sequence
  Description of Artificial Sequence Synthetic peptide
QIQLVQSGPELKKPGASVRVSCKASGYSFT

SEQ ID NO: 174 - protein, 14 aa, Artificial Sequence
  Description of Artificial Sequence Synthetic peptide
WVRQSPGKGLEWIG

SEQ ID NO: 175 - protein, 32 aa, Artificial Sequence; identical to SEQ ID NO: 39
  Description of Artificial Sequence Synthetic peptide

SEQ ID NO: 176 - protein, 11 aa, Artificial Sequence
  Description of Artificial Sequence Synthetic peptide
WGQGTTVTVSS

SEQ ID NO: 177 - protein, 30 aa, Artificial Sequence
  Description of Artificial Sequence Synthetic peptide
QIQLVQSGPELVKPGASVRVSCKASGYSFT

SEQ ID NO: 178 - protein, 14 aa, Artificial Sequence
  Description of Artificial Sequence Synthetic peptide
WVRQSPGKGLEWIG

SEQ ID NO: 179 - protein, 32 aa, Artificial Sequence; identical to SEQ ID NO: 39
  Description of Artificial Sequence Synthetic peptide

SEQ ID NO: 180 - protein, 11 aa, Artificial Sequence
  Description of Artificial Sequence Synthetic peptide
WGQGTTVTVSS

SEQ ID NO: 181 - protein, 30 aa, Artificial Sequence
  Description of Artificial Sequence Synthetic peptide
QIQLVQSGPEVVKPGASVRVSCKASGYSFT

SEQ ID NO: 182 - protein, 14 aa, Artificial Sequence
  Description of Artificial Sequence Synthetic peptide
WVRQSPGKGLEWIG

SEQ ID NO: 183 - protein, 32 aa, Artificial Sequence; identical to SEQ ID NO: 39
  Description of Artificial Sequence Synthetic peptide

SEQ ID NO: 184 - protein, 11 aa, Artificial Sequence
  Description of Artificial Sequence Synthetic peptide
WGQGTTVTVSS

SEQ ID NO: 185 - protein, 5 aa, Mus musculus
DYNVY

SEQ ID NO: 186 - protein, 17 aa, Mus musculus
YIDPYNGITIYDQNFKG

SEQ ID NO: 187 - protein, 8 aa, Mus musculus
DVTTALDF

SEQ ID NO: 188 - protein, 5 aa, Artificial Sequence
  Description of Artificial Sequence Synthetic peptide
DYNVY

SEQ ID NO: 189 - protein, 17 aa, Artificial Sequence
  Description of Artificial Sequence Synthetic peptide
YIDPYNGITIYDQNLKG

SEQ ID NO: 190 - protein, 8 aa, Artificial Sequence
  Description of Artificial Sequence Synthetic peptide
DVTTALDF

SEQ ID NO: 191 - protein, 107 aa, Homo sapiens [sequence omitted]

SEQ ID NO: 192 - protein, 332 aa, Homo sapiens
EFASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 193 - protein, 107 aa, Homo sapiens [sequence omitted]

SEQ ID NO: 194 - protein, 329 aa, Homo sapiens
EFASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 195 - protein, 14 aa, Mus musculus
WVRQSHGKSLEWIG

SEQ ID NO: 196 - protein, 14 aa, Homo sapiens
WVRQSPGKGLEWIG

(19) United States
(12) Patent Application Publication
(10) Pub. No.: US 2013/0011389 A1
(43) Pub. Date: Jan. 10, 2013

(54) COMPOSITIONS AND METHODS FOR TREATING COAGULATION RELATED DISORDERS

(76) Inventors: Jin-An JIAO, Fort Lauderdale, FL (US); Hing C. Wong, Weston, FL (US); Jack O. Egan, Plantation, FL (US)

(21) Appl. No.: 13/327,659

(22) Filed: Dec. 15, 2011

Related U.S. Application Data
(63) Continuation of application No. 13/013,767, filed on Jan. 25, 2011, now abandoned.
Application No. 13/013,767 is a continuation of application No. 12/036,188, filed on Feb. 22, 2008, now abandoned.
Application No. 12/036,188 is a continuation of application No. 11/311,702, filed on Dec. 19, 2005, now abandoned.
Application No. 11/311,702 is a continuation of application No. PCT/US04/17900, filed on Jun. 4, 2004.
(60) Provisional application No. 60/538,892, filed on Jan. 22, 2004.
(60) Provisional application No. 60/480,254, filed on Jun. 19, 2003.

(51) Int. Cl. A61K 39/395; A61P 11/00

(52) U.S. Cl. 424/133.1

(57) ABSTRACT

Disclosed are methods for preventing or treating sepsis, a sepsis-related condition or an inflammatory disease in a mammal. In one embodiment, the method includes administering to the mammal a therapeutically effective amount of at least one humanized antibody, chimeric antibody, or fragment thereof that binds specifically to tissue factor (TF) to form a complex in which factor X or factor IX binding to the complex is inhibited and the administration is sufficient to prevent or treat the sepsis in the mammal. The invention has a wide spectrum of useful applications including treating sepsis, disorders related to sepsis, and inflammatory diseases such as arthritis.

### CROSS REFERENCE TO RELATED APPLICATIONS

This application is a continuation of U.S. patent application Ser. No. 12/036,188, filed Feb. 22, 2008, which is a continuation of U.S. patent application Ser. No. 11/311,702, filed Dec. 19, 2005, which is a continuation of International Patent Application No. PCT/US04/17900, filed Jun. 4, 2004, which claims priority benefit to U.S. Provisional Patent Application No. 60/538,892, filed Jan. 22, 2004 and U.S. Provisional Patent Application No. 60/480,254, filed Jun. 19, 2003, the disclosures of all of which are hereby incorporated by reference herein in their entirety.

### FIELD OF THE INVENTION

The present invention features compositions and methods for preventing or treating disorders that relate to undesired activation of blood coagulation. In some instances, the coagulation contributes to certain inflammatory diseases and related disorders. In one aspect, the invention provides methods for treating such disorders by administering a therapeutically effective amount of a chimeric or humanized antibody that binds tissue factor (TF) specifically. The invention has a wide spectrum of important applications including use in the prevention or treatment of inflammation including sepsis and arthritis.

### BACKGROUND OF THE INVENTION

There is increasing recognition of relationship between coagulation and inflammation. For instance, certain coagulation factors are thought to activate pro-inflammatory cells and elicit inflammatory responses. On the other hand, some pro-inflammatory cytokines have been reported to induce TF expression and generate coagulation factors. Studies in certain primates support existence of the relationship by showing that certain anticoagulants reduce inflammation. See F. B. Taylor Jr. et al., J. Clin. Invest. 79:918-925 (1987); M. Levi et al., J. Clin. Invest. 93:114-120 (1994); and M. C. Minnema et al., Blood 95:1117-23 (2000).

Much has been reported about blood coagulation. For instance, thrombin is a blood protein that is believed to provide a link between coagulation and inflammation. Most thrombin is generated by TF-initiation of the coagulation cascade. Other key coagulation factors include Factor VIIa and Factor Xa. Thrombin is thought to occupy multiple roles in pro-coagulant, anticoagulant, inflammatory, and mitogenic responses. See generally L. Styer, Biochemistry, 3rd Ed., W.H. Freeman Co., New York; and A. G. Gilman et al., in The Pharmacological Basis of Therapeutics, 8th Ed., McGraw-Hill Inc., New York.

Certain infectious agents are thought to disturb the TF-initiated coagulation cascade. Fortunately, localized activation of the coagulation cascade often keeps the disturbance in check. Sometimes however, aberrant TF expression leads to serious and potentially life-threatening thrombotic disorders. For example, it has been suggested that bacterial induction of TF expression can lead to sepsis, disseminated intravascular coagulation (DIC), widespread fibrin deposition, and other complications. The increase in TF expression is thought to be an important factor in facilitating progression of these disorders. An example of progression of an inflammatory disorder is the progression of acute lung injury (ALI) to acute respiratory distress syndrome (ARDS) followed by progressive injury involving additional organ systems, such as the kidneys, leading to progressively more severe forms of sepsis. See Welty-Wolf, K. et al. Am. J. Respir. Crit. Care Med. 164:1988 (2001), for instance.

There have been attempts to understand coagulation related disorders such as sepsis.

For instance, sepsis is a term that has used to characterize a spectrum of clinical conditions facilitated by a host immune response to infection, trauma, or both. Sepsis has been characterized as an uncontrolled cascade of blood coagulation, fibrinolysis, and inflammation. At certain steps in the cascade, an auto-amplification process contributes to an acceleration of coagulation abnormalities, undesired inflammation, and endothelial injury. The amplification process is the result of inflammatory cytokines up-regulating the expression of TF on cells such as endothelial cells and monocytes, resulting in increased activation of the coagulation cascade, which in turn results in the activation of PAR receptors and the up-regulation of the production of inflammatory cytokines. See Osterud, B. et al., Thromobsis Hemost. 83:861 (2000); Mechtcheriakova, D. et al., FASEB J. 15:230 (2001); Shen, B. Q. et al., J. Biol. Chem. 276:5281 (2001).

More specifically, sepsis has been characterized by systemic activation of inflammation and blood coagulation. Fibrinolyisis can be suppressed. Some research has pointed to a hemostatic imbalance that is thought to promote e.g., DIC and microvascular thrombosis. These and related indication are believed to impact normal organ function which can lead to death.

Sepsis and related disorders continue to be linked to patient contact with hospitals and out-patient clinics. Accordingly, managing these disorders is a prime concern of many health administrators, clinicians and insurers.

There are reports that undesired activation of blood coagulation is a key feature of certain inflammatory diseases. For example, extravascular fibrin deposition has been found in the autoimmune lesions of patients with rheumatoid arthritis (RA), glomerulonephritis, multiple sclerosis, psoriasis, Sjogren's syndrome and inflammatory bowel disease. See Weinberg, et al., Arthritis Rheum. 34:996-1005 (1991); Wakefield, et al., J Clin Pathol 47:129-133 (1994); Schoph, et al., Arch Dermatol Res 285:305-309 (1993); Zeher, et al., Clin Immunol Immunopathol 71:149-155 (1994); More, et al., J Clin Pathol 46:703-708 (1993).

Moreover, elevated TF levels are thought to be associated with systemic lupus erythematosus. See Segal, et al., J Rheumatol 27:2827-2832 (2000). See also Nakano, et al., Clin Exp Rheumatol 17:161-170 (1999); Morris, et al., Ann Rheum Dis 53:72-79 (1994); Furmaniak-Kazmierczak, et al., J Clin Invest 94:472-480 (1994); Bokarewa, et al., Inflamm Res 51:471-477 (2002).

There have been suggestions that certain inflammatory conditions may be treated by blocking thrombosis. See R. Gordon et al., The New England Journal of Medicine 344:699-709, 759-762 (2001). Certain anti-TF antibodies have been reported to be of some help in reducing some types of inflammation. See Levi, M. et al., supra.

There have been efforts to develop antibodies that bind blood coagulation factors.

For instance, U.S. Pat. Nos. 5,986,065 and 6,555,319 to Wong, H. et al. and PCT/US98/04644 (WO 98/40408) disclose a variety of such antibodies. Specifically provided are murine antibodies, chimeric antibody derivatives and fragments thereof with significant binding affinity and specificity for tissue factor (TF). Use of chimeric antigen binding molecules are believed to reduce risk of an undesired immune response in human patients. See also S. L. Morrison and V. Oi, Adv. Immunol. 44:65 (1989) (reporting methods of making human-mouse chimeric antibodies).

A variety of approaches have been used to make antibodies more immunologically acceptable to humans. Some use recombinant DNA technologies. For instance, one strategy has been to clone and modify non-human antibodies to more closely resemble human antibodies. Collectively, such antibodies have been referred to as “humanized”. See U.S. Pat. Nos. 5,766,886 to Studnicka et al.; 5,693,762 to Queen et al.; 5,985,279 to Waldeman et al.; 5,225,539 to Winter; 5,639,641 to Pedersen, et al.; and references cited therein for methods of making and using humanized antibodies.

Additional strategies for making humanized antibodies have been reported. See E. Padlan Mol. Immunol. 28:489 (1991); Jones et al., Nature 321:522-525 (1986); Junghans et al., supra; and Roguska, et al., PNAS (USA) 91:969 (1994). See also published U.S. Patent Applications 2003/0109860 A1 and 2003/0082636.

It is unclear if prior antibodies are robust enough to block or reduce undesired activation of blood coagulation. More specifically, it is unclear if such antibodies are potent enough to prevent or treat sepsis and inflammatory diseases such as arthritis.

### SUMMARY OF THE INVENTION

The present invention features compositions and methods for preventing or treating disorders relating to undesired activation of blood coagulation. In one aspect, the invention provides methods for preventing or treating such disorders by administering to a mammal a therapeutically effective amount of a chimeric or humanized antibody that binds tissue factor (TF). The invention has a wide spectrum of important applications including use in the treatment of sepsis and inflammatory diseases such as arthritis.

We have found that antibodies and antigen binding fragments thereof that specifically bind an epitope predominant to native human TF are suitable for preventing or treating disorders relating to undesired activation of blood coagulation. Preferred antibodies and fragments specifically bind native human TF and do not substantially bind non-native or denatured TF. More particular antibodies and fragments suitable for use with the present invention bind human TF so that at least one of Factor X (FX) and Factor IX (FIX) do not effectively bind to the TF-Factor VIIa complex. Additionally preferred antibodies and fragments reduce. or block TF function, typically by reducing or blocking FX binding or gaining access to TF molecules. Further preferred antibodies and fragments suitable for use with the invention do not significantly inhibit or block interaction or binding between TF and Factor VIIa, or inhibit or block activity of a TF-Factor VIIa complex with respect to materials other than FX or FIX.

As discussed, it is believed that undesired activation of blood coagulation underlies sepsis and a variety of specific inflammatory diseases. More specifically, unwanted TF-mediated coagulation is thought to initiate and/or prolong such disorders in many cases. It is thus an object of the present invention to provide antibodies and fragments thereof that specifically bind TF to reduce or inactivate many if not all TF-associated functions. Such functions include, but are not limited to, blocking or inhibiting at least one of FIX and FX binding to the TF complex. Without wishing to be bound to theory, it is believed that by blocking or inhibiting binding of at least one of those factors to the TF complex, activation of unwanted blood coagulation can be reduced or in some cases eliminated. That is, by blocking or inhibiting such unwanted processes according to the invention, it is believed that it is possible to prevent, treat or alleviate symptoms associated with sepsis and specific inflammatory diseases.

Preferably, such antibodies and fragments thereof are chimeric or humanized and are generally suitable for use in primates and particularly human patients in need of treatment.

There is recognition that sepsis and related conditions result from a potent and potentially life-threatening immune response to infection, trauma, or both. Typically, blood throughout the vasculature is subject to coagulation, resulting in complications such as inflammation, disseminated intravascular coagulation (DIC), clotting, and organ distress. Death can ensue in a matter of hours or less unless the condition is treated rapidly. Prior to the present invention, it was not clear if any anti-TF antibody or TF-binding fragment would be robust enough to prevent or treat sepsis and related conditions.

However, it has been found that the anti-TF binding antibodies described herein are robust enough (i.e., bind human TF specifically enough and with appropriate avidity) to inhibit or block unwanted activation of the coagulation cascade. It has also been found that such activity is beneficial and can be used to prevent or treat sepsis and related conditions. As discussed below, we have found that such antibodies and fragments show good attenuation of inflammation, disseminated intravascular coagulation (DIC), clotting, organ distress and related conditions in an in vivo animal model of human sepsis.

Such potent blocking or inhibition of the blood coagulation cascade has also been found to be highly useful in the prevention or treatment of certain inflammatory diseases. Without wishing to be bound to theory, it is believed that by using the invention to block or reduce undesired activation of blood coagulation, it is possible to prevent, treat or alleviate symptoms associated with one or a combination of the inflammatory diseases. Importantly, the anti-TF binding antibodies described herein have been found to be robust enough to inhibit or block unwanted activation of the coagulation cascade, thereby helping to prevent, treat or alleviate symptoms associated with arthritis and other inflammatory diseases.

Accordingly, and in one aspect, the invention provides a method for preventing or treating at least one of sepsis and an inflammatory disease in a mammal. In one embodiment, the method includes administering to the mammal a therapeutically effective amount of at least one humanized antibody, chimeric antibody, or fragment thereof that binds specifically to human TF to form a complex. A more preferred antibody reduces or blocks at least one of FX and FIX binding to the complex. Practice of the method is highly useful for preventing, treating, or reducing the severity of symptoms associated with sepsis and inflammatory diseases including, but not limited to, rheumatoid arthritis (RA).

The present invention provides other important uses and advantages.

For instance, we have discovered that preferred humanized antibodies, chimeric antibodies and fragments thereof desirably block at least one of FX and FIX binding to the TF-FVIIa complex. Preferably, such antibodies and fragments also inhibit or block FX or FIX activation by the complex. Surprisingly, such antibodies, when tested in the in vivo animal of human sepsis provided herein, are robust enough to prevent, treat or alleviate symptoms of sepsis and related complications. Also surprisingly, such preferred antibodies and fragments are robust enough to prevent, treat, or alleviate symptoms associated with particular inflammatory diseases. Importantly, preferred use of the present invention has identified and takes advantage of a particular immunological target (epitope) on the human TF molecule that can be exploited to prevent, treat, or alleviate symptoms associated with these indications.

The invention is flexible and can be used in a variety of settings in which sepsis and inflammatory diseases may be suspected or predominate.

For instance, and in one embodiment, the invention can be used in hospitals, clinics and other medical settings where sepsis has become a major health problem. Especially problematic has been emergence of antibiotic resistance microorganisms such as bacteria which, if present in the blood, can rapidly produce septic shock and related complications. Practice of the invention can be used to hold the sepsis or related condition at bay while caregivers identify an appropriate treatment protocol, or perhaps reverse the effects of sepsis. It is thus anticipated as an object of the invention to provide sepsis prevention or treatment methods in which administration of the antibodies and fragments disclosed herein may be combined with administration of one or more antibiotics.

The invention finds further use in emergency medical settings (e.g., ambulance, combat) in which the prevention and treatment methods disclosed herein can be administered at the point of care. Thus in one invention embodiment, the sepsis or sepsis-related condition can be held under some control while the patient is transported to a hospital or clinic for evaluation and treatment.

In other invention embodiments, the invention can be used to prevent, treat or reduce symptoms associated with arthritis and especially rheumatoid arthritis. For instance, by blocking or reducing the unwanted activation of blood coagulation, it is now possible to reduce the painful inflammation, pathological tissue destruction and remodeling typically associated with arthritis and related inflammatory diseases.

In another aspect, the invention provides a kit for performing the methods of this invention. In one embodiment, the kit includes at least one humanized antibody, chimeric antibody, or fragment thereof provided herein.

The invention also provides a method for reducing cytokine production in a mammal. In one embodiment, the method includes administering to the mammal a therapeutically effective amount of at least one humanized antibody, chimeric antibody, or fragment thereof that binds specifically to tissue factor (TF) to form a complex. Preferably, Factor X or Factor IX binding to the complex is inhibited and the administration is sufficient to reduce the cytokine production in the mammal. Suitable humanized antibodies, chimeric antibodies and fragments thereof are disclosed above and in the discussion and examples that follow.

Further provided by the present invention is a method for preventing or treating a sepsis-related condition in a mammal. In one embodiment, the method includes administering to the mammal a therapeutically effective amount of at least one humanized antibody, chimeric antibody, or fragment thereof that binds specifically to tissue factor (TF) to form a complex. Preferred antibodies and fragments are described herein and include those in which Factor X or factor IX binding to the complex is inhibited. Preferably, administration is sufficient to prevent or treat the condition in the mammal.

The invention further provides a method for preventing or treating sepsis-induced anemia in a mammal. In one embodiment, the method includes administering to the mammal a therapeutically effective amount of at least one humanized antibody, chimeric antibody, or fragment thereof that binds specifically to tissue factor (TF) to form a complex. Preferred antibodies and fragments are disclosed herein including those in which Factor X or Factor IX binding to the complex is inhibited. Preferably, the administration is sufficient to prevent or treat the condition in the mammal.

Other aspects of the invention are discussed infra.

### BRIEF DESCRIPTION OF THE DRAWINGS

FIGS. 1A and 1B shows the nucleic acid (SEQ ID NOS: 1 and 3) and amino acid (SEQ ID NOS: 2 and 4) sequences of light chain and heavy chain variable domains of H36.D2.B7, the murine anti-tissue factor antibody, with hypervariable regions (CDRs or Complementarity Determining Regions) underlined (single underline for nucleic acid sequences and double underline for amino acid sequences).

FIG. 2 is a drawing showing a plasmid map of humanized anti-TF IgG1 antibody expression vector (pSUN-34).

FIGS. 3A-D are sequences of partially and fully humanized light chain (LC) variable domains of the anti-TF antibody (SEQ ID NO.: ______). FIG. 3A shows the sequence named “LC-09” which is representative of a fully humanized LC framework (SEQ ID NO.: ______). Light chain CDR sequences of cH36 and LC-09 are shown in FIGS. 3B-D (SEQ ID NOS.: ______, respectively).

FIGS. 4A-D are drawings showing the sequences of partially and fully humanized heavy chain (HC) variable domains of the anti-TF antibody (SEQ ID NOS.: ______). FIG. 4A shows the sequence named “HC-08” which is representative of a fully humanized HC framework (SEQ ID NO.: ______). Heavy chain CDR sequences for cH36 and HC-08 are shown in FIGS. 4B-D (SEQ ID NOS.: ______ respectively).

FIGS. 5A-B are sequences showing human constant domains in the IgG1 anti-tissue factor antibody (hOAT), with FIG. 5A showing the human kappa light chain constant domain (SEQ ID NO.: ______) and FIG. 5B showing the human IgG1 heavy chain constant domain (SEQ ID NO.: ______). The figures show hOAT (IgG1) constant domain amino acid sequences.

FIGS. 6A-B are sequences showing human constant domains in the IgG4 anti-tissue factor antibody (hFAT) with FIG. 6A showing the human kappa light chain constant domain (SEQ ID NO.: ______) ______) and FIG. 6B showing the human IgG4 heavy chain constant domain (SEQ ID NO.: ______).

FIGS. 7A-D are graphs showing a change in plasma IL-6 and IL-8 (FIGS. 7A-B) concentrations (FIGS. 7A-B); or IL-1β and TNF-α concentrations (FIGS. 7C-D) in rhesus monkeys following an infusion of live E. coli in a lethal sepsis model.

FIGS. 8A-C are graphs showing that cH36 attentuates sepsis-induced acute lung injury (ALI). In the figures, AaDO₂ is in mmHg, time in hours, pulmonary system compliance (Cst) in ml/cm water, and pulmonary arterial pressure (PAM) in mmHg.

FIGS. 9A-B are graphs showing Kidney Myeloperoxidase (A) and Small Bowel Wet/Dry Weight Ratio (B) in Baboons.

FIG. 10A-D are graphs showing that cH36 attenuates sepsis and related conditions in baboons. cH36 attenuates sepsis-induced coagulopathy (fibrinogen in mg/DL; time in hours; partial prothrombin time (PTT) in seconds; and TAT in micrograms/L).

FIGS. 11A-B are graphs showing that elevations in serum IL-8 (A) and IL-6(B) are attenuated by treatment with cH36.

FIG. 12 is a graph showing that elevations in bronchial alveolar levage IL-8, IL-6 and TNFR1 levels attenuated by treatment with cH36.

FIGS. 13A-C are graphs showing mean urine output (13A), mean blood pH (13B), and serum bicarbonate levels (13C) in baboons treated with control and cH36 antibodies.

### DETAILED DESCRIPTION OF THE INVENTION

As discussed, the invention provides a method for preventing, treating, or alleviating symptoms associated with sepsis or inflammatory diseases such as arthritis. Practice of the method involves administering to a mammal in need of such treatment a therapeutically effective amount of at least one of a humanized antibody, chimeric antibody, or human TF-binding fragment to prevent or treat these diseases and related conditions.

There have been efforts to understand the etiology of sepsis and conditions related to sepsis. For instance, there is recognition that early events in the sepsis cascade are triggered by the host's immune response, thereby facilitating damaging actions on the vascular endothelium. Subendothelial structures are exposed and collagenases are liberated. Endothelial cells expressing tissue factor (TF) are exposed, triggering the extrinsic pathway for activation of the coagulation cascade and accelerating the production of thrombin. Concurrently, the endothelial damage causes further exacerbation of inflammation, resulting in neutrophil activation, neutrophil-endothelial cell adhesion, and further elaboration of inflammatory cytokines. These inflammatory processes further contribute to vascular endothelial dysfunction. Endogenous modulators of homeostasis, such as protein C and anti-thrombin III (AT III), are consumed and their levels become deficient as the body attempts to return to a normal functional state. Under normal conditions, the endothelial surface proteins thrombomodulin and endothelial protein C receptor (EPCR), activate protein C and its modulating effects. In sepsis, the endothelial damage impairs this function of thrombomodulin and EPCR, thereby contributing to the loss of control. Left unopposed, the endothelial damage accumulates. This uncontrolled cascade of inflammation and coagulation fuels the progression of sepsis, resulting in hypoxia, widespread ischemia, organ dysfunction, and ultimately death for a large number of patients.

By the phrase “sepsis-related condition” is meant those known to precede, accompany, or follow sepsis including, but not limited to, disseminated intravascular coagulation (DIC), fibrin deposition, thrombosis, and lung injury, including acute lung injury (ALI), or acute respiratory distress syndrome (ARDS). A particular type of lung injury amenable to prevention or treatment by use of the invention is sepsis-induced acute lung injury. See Welty-Wolf, et al., Am. J. Respir. Crit. Care Med. 164:1988 (2001), for instance. Also encompassed are certain renal disorders accompanying sepsis such as acute tubular necrosis (ACN) and related conditions.

Acute lung injury (ALI) and acute respiratory distress syndrome (ARDS) are severe disorders that continue to receive significant attention. In particular, an important feature of ALI and ARDS, is local activation of extrinsic coagulation and inhibition of fibrinolysis. As the injury evolves, these perturbations have been reported to cause deposition of fibrin in the microvascular, interstitial, and alveolar spaces of the lung leading to capillary obliteration and hyaline membrane formation. Components of the extrinsic coagulation pathway (e.g. tissue factor (TF), thrombin, and fibrin) signal alterations in inflammatory cell traffic and increases in vascular permeability. Procoagulants and fibrin are also thought to promote other key events in the injury including complement activation, production of pro-inflammatory cytokines, inhibition of fibrinolysis, and remodeling of the injured lung. Without wishing to be bound to theory, it is believed that by reducing or blocking these initiating events (extrinsic coagulation) and downstream effects (pro-inflammatory events) in the lungs, disordered fibrin turnover can be reduced or blocked and the evolution of severe structural and functional injury can be reduced or averted during ALI/ARDS. Coagulation blockade can target the TF-Factor VIIa (TF-FVIIa) complex at several points, but the effects of these different strategies on inflammation and the development of lung injury are still being confirmed. The Examples show use of a chimerized monoclonal antibody against human TF (cH36) and its Fab fragment (cH36-Fab) in blocking initiation of coagulation in gram-negative sepsis and prevent acute lung injury.

The present invention is intended, in one embodiment, to prevent or treat sepsis and sepsis related conditions (such as DIC, ALI and ARDS) by reducing or blocking activity of a key component of the blood coagulation cascade (i.e., TF). Preferred humanized antibodies, chimeric antibodies, and fragments thereof specifically bind human TF typically to block at least one of FX or Factor IX binding to the TF complex. Typically, such preferred antibodies and fragments also inhibit or block FX or FIX activation of that complex. Thus, the compositions and methods of the invention reduce or block unwanted activation of the blood coagulation cascade by decreasing or preventing activity of a key molecular component.

Such preferred antibodies are different from prior antibodies such as those provided by U.S. Pat. No. 6,274,142 (“'42”) to O'Brien et al. For instance, the '142 patent reports TF neutralizing antibodies that cannot bind to Factor VII/VIIa or effect proteolysis of Factors IX or X. In contrast, preferred antibodies of the invention substantially reduce or block at least one of FX or FIX binding to the TF complex. See also PCT/US01/07501 (WO 01/70984).

As mentioned previously, unwanted coagulation activation is a prominent feature of certain inflammatory diseases, particularly those associated with autoimmunity. See Weinberg, et al., Arthritis Rheum. 34:996-1005 (1991); Kincaid-Smith, Kidney Int 7:242-253 (1975); Wakefield, et al., J Clin Pathol 47:129-133 (1994); Schoph, et al., Arch Dermatol Res 285:305-309 (1993); Zeher, et al., Glin Immunol Immunopathol 71:149-155 (1994); More, et al., J Clin Pathol 46:703-708 (1993). Elevated TF levels have been reported to be associated with disease activity in systemic lupus erythematosus. See Segal, et al., J Rheumatol 27:2827-2832 (2000). It is thus an object of this invention to help control tissue factor-mediated coagulation, thereby reducing or in some instances blocking the inflammation, pathological tissue destruction and undesired remodeling that is a hallmark of many inflammatory diseases.

By the phrase “inflammatory disease” including plural forms is meant a pathological condition associated with an unwanted TF-mediated activation of the coagulation. Preferred inflammatory diseases are usually associated with a known or suspected autoimmune condition. Typically, such diseases are further associated with enhanced production of pro-inflammatory cytokines and/or chemokines as determined by standard methods. Examples of such cytokines include IL-1, TNFα, GM-CSF, M-CSF, IL-6, LIF, IL-15, IFNα, and IL12. Examples of such chemokines include IL-8, MIP-1α, MCP-1, ENA-78, and RANTES. Typically, but not exclusively, one or more of neovascularization and extravascular fibrin deposition is associated with the inflammatory disease. More particular examples of inflammatory diseases according to the invention include arthritis, preferably rheumatoid arthritis (RA); glomerulonephritis, multiple sclerosis, psoriasis, Sjogren's syndrome and inflammatory bowel disease. Arthritis can be readily detected by one or a combination of features including presence of synovial inflammation, pannus formation, and cartilage destruction.

Preferred use of the invention will help reduce, prevent or alleviate symptoms of the inflammatory disease typically by decreasing TF-mediated coagulation activation. Such activation by TF is believed to provide many disadvantages including, but not limited to, supporting the production of inflammatory molecules, enhancing proinflammatory cell activity, increasing tissue destruction; increasing unwanted remodeling and boosting angiogenesis. The invention thus provides a new and fundamental means for addressing inflammatory disease by providing compositions and methods for specifically binding and inhibiting function of TF.

Practice of the invention will particularly help in the prevention and treatment of inflammatory autoimmune diseases. Recent work is in agreement with this inventive concept. See Marty, et al., J Clin Invest 107:631-640 (2001); Varisco, et al., Ann Rheum Dis 59:781-787 (2000); Buso, et al., Arthritis Rheum 48:651-659 (2003).

As discussed, the U.S. Pat. Nos. 5,986,065 and 6,555,319 to Wong, H. et al. and PCT/US98/04644 (WO 98/40408) disclose a variety of murine anti-TF antibodies and antigen binding fragments with good human TF binding characteristics. Such antibodies and fragments can be employed in accord with the present invention. Additionally, such antibodies and fragments can be used to treat experimentally induced sepsis, for instance, in a relevant rodent model. However, and as will be appreciated, such murine antibodies and fragments are not usually appropriate for use in other mammals such as primates and especially human subjects. Further suitable antibodies and fragments are disclosed by U.S. Patent Application Publication No. 20030082636; WO 03/037911 and WO 98/40408.

By the phrase “antigen binding fragment” is meant at least a part of an antibody that specifically binds antigen. An example of such a fragment includes an antibody V domain. Examples of a suitable V domain binding partner include a C domain and acceptable fragments thereof. Further suitable fragments include parts of the V domain having a combined molecular mass for the V domain of between from about 15 kilodaltons to about 40 kilodaltons, preferably between from about 20 kilodaltons to about 30 kilodaltons, more preferably about 25 kilodaltons as determined by a variety of standard methods including SDS polyacrylamide gel electrophoresis or size exclusion chromatography using appropriately sized marker fragments, mass spectroscopy or amino acid sequence analysis. Further specific antigen binding fragments include Fab, F(v), Fab′, F(ab′)₂ and certain single-chain constructs that include the antibody V domain.

Additionally suitable antigen binding fragments include at least part of an antigen binding V domains alone or in combination with a cognate constant (C) domain or fragment thereof (“cognate” is used to denote relationship between two components of the same immunoglobulin heavy (H) or light (L) chain). Typical C domain fragments have a molecular mass of between from about 5 kilodaltons to about 50 kilodaltons, more preferably between from about 10 kilodaltons to about 40 kilodaltons, as determined by a variety of standard methods including SDS polyacrylamide gel electrophoresis or size exclusion chromatography using appropriately sized marker fragments, mass spectroscopy or amino acid sequence analysis. Additionally suitable antigen binding fragments are disclosed below as well as in U.S. Pat. Nos. 5,986,065 and 6,555,319 to Wong, H. et al. and PCT/US98/04644 (WO 98/40408). See also U.S. Patent Application Publication No. 20030082636 and the following International Applications: WO 03/037911 and WO 98/40408.

As also mentioned, one approach to minimize any potential immunorejection by primate hosts such as human patients is to make a chimeric antibody. By the phrase “chimeric antibody” or related phrase including plural forms is meant antibodies as disclosed herein whose light and heavy chain genes have been constructed, typically by genetic engineering, from immunoglobulin gene segments belonging to different species, usually a primate and preferably a human. For example, the variable (V) domains of the genes from a mouse antibody such as H36 may be joined to human constant (C) domains, such as γ₁, γ₂, γ₃, or γ₄. A typical therapeutic chimeric antibody is thus a hybrid protein consisting of the V or antigen-binding domain from a mouse antibody and the C or effector domain from a human antibody, although other mammalian species may be used. A specifically preferred chimeric antibody for use with the invention is the anti-tissue factor antibody cH36 disclosed below in the Examples.

Suitable chimeric antibodies for use with the invention can be made by one or a combination of known strategies. As disclosed in the U.S. Pat. Nos. 5,986,065, 6,555,319 and PCT/US98/04644 (WO 98/40408), a highly useful murine anti-TF antibody can be readily obtained from a variety of sources including the American Type Culture Collection (ATCC, 10801 University Boulevard, Manassas, Va. 20110). The antibody has been deposited as ATCC Accession No. HB-12255. Alternatively, suitable antibodies can be made de novo (as polyclonal or monoclonal as needed) in accord with procedures disclosed in the U.S. Pat. Nos. 5,986,065 and 6,555,319 for instance.

See also U.S. Patent Application Publication No. 20030082636 and the following International Applications: WO 03/037911 and WO 98/40408.

The antibody deposited with the ATCC H36 is referred to herein as H36. It is also referenced as H36.D2 and as H36.D2.B7. The antibody designated as H36 is the antibody produced by the mother clone, and H36.D2 is obtained from the primary clone, whereas H36.D2.B7 is obtained from the secondary clone. No differences were observed between the antibody produced by those three clones with respect to the antibody's ability to inhibit TF or other physical properties. In general usage, H36 is often used to indicate anti-TF antibody produced by any of these clones or related cell lines producing the antibody. The mouse-human chimeric version of H36 is referred to cH36 (and also as Sunol-cH36). See also the U.S. Pat. No. 5,986,065 and PCT/US98/04644 (WO 98/40408) for more specific information about the H36 antibody.

A preferred chimeric antibody combines the murine variable domain from a suitable antibody such as H36 and a human constant domain. The manipulation is usually achieved by using standard nucleic acid recombination techniques. A variety of types of such chimeric antibodies can be prepared, including e.g. by producing human variable domain chimeras, in which parts of the variable domains, especially conserved regions of the antigen-binding domain, are of human origin and only the hypervariable regions are of non-human origin. See S. L. Morrison, Science, 229:1202-1207 (1985); Oi et al., BioTechniques 4:214 (1986); Teng et al., Proc. Natl. Acad. Sci. U.S.A., 80:7308-7312 (1983); Kozobor et al., Immunology Today 4:72-79 (1983); Olsson et al., Meth. Enzymol. 92:3-16 (1983); U.S. Pat. No. 5,986,065; and PCT/US98/04644 (WO 98/40408).

Nucleic acids encoding the H36 variable and constant regions have been disclosed in the U.S. Pat. No. 5,986,065; and PCT/US98/04644 (WO 98/40408), for example. See also U.S. Patent Application Publication No. 20030082636; WO 03/037911 and WO 98/40408.

In one embodiment, the anti-TF chimeric antibody will include a human light chain constant (C) domain i.e., Cκ, Cλ, or a fragment thereof. Often, the humanized light chain fragment will have an amino acid length of between from about 80 to about 250 amino acids, preferably between from about 95 to about 235 amino acids, more preferably between from about 104 to about 225 amino acids. The size of the humanized light chain fragment can be determined by a variety of standard methods including SDS polyacrylamide gel electrophoresis or size exclusion chromatography using appropriately sized marker fragments, mass spectroscopy or amino acid sequence analysis.

Typically, the chimeric antibody for use in the present methods further includes a human heavy chain variable (V) domain having an amino acid length of between about 80 to about 650 amino acids, preferably between from about 95 to about 540 amino acids, more preferably about 102 to about 527 amino acids as determined e.g., by standard SDS polyacrylamide gel electrophoresis or size exclusion chromatography using appropriately sized marker fragments; mass spectroscopy or amino acid sequence analysis.

Nucleic acid sequence encoding suitable human light chain C and V domains have been reported. See e.g., Kabat et al. in Sequences of Proteins of Immunological Interest Fifth Edition, U.S. Dept. of Health and Human Services, U.S. Government Printing Office (1991) NIH Publication No. 91-3242; and GenBank. See the National Center for Biotechnology Information (NCBI)-Genetic Sequence Data Bank (Genbank) at the National Library of Medicine, 38A, 8N05, Rockville Pike, Bethesda, Md. 20894. See Benson, D. A. et al., Nucl. Acids. Res. 25:1 (1997) for a more specific description of Genbank.

Suitable recombinant techniques for use in making the chimeric antibodies and other antibodies and fragments as reported herein have been disclosed. See generally Sambrook et al. in Molecular Cloning: A Laboratory Manual (2d ed. 1989); and Ausubel et al., Current Protocols in Molecular Biology, John Wiley & Sons, New York, (1989).

For some invention applications in which a minimal immunoresponse against an anti-TF antibody is needed, it will be useful to prepare humanized antibodies. By the phrase “humanized” is meant an immunoglobulin that includes at least one human FR subset, preferably at least two or three of same, more preferably four human FR subsets, and one or more CDRs from a non-human source, usually rodent such as a rat or mouse immunoglobulin. Typically preferred humanized immunoglobulins of the invention will include two or more preferably three CDRs. Constant domains need not be present but are often useful in assisting function of humanized antibodies intended for in vivo use. Preferred constant domains, if present, are substantially identical to human immunoglobulin constant domains i.e., at least about 90% identical with regard to the amino acid sequence, preferably at least about 95% identical or greater. Accordingly, nearly all parts of the humanized immunoglobulin, with the possible exception of the CDRs, are preferably substantially identical to corresponding parts of naturally occurring human immunoglobulin sequences.

Methods for determining amino acid sequence identity are standard in the field and include visual inspection as well as computer-assisted approaches using BLAST and FASTA (available from the National Library of Medicine (USA) website). Preferred matching programs for most embodiments are available from website for the international ImMunoGeneTics (IMGT) database and a more preferred matching program for this embodiment is the program called Match which is available in the Kabat database. See Johnson G, Wu T. “Kabat database and its application: Future directions.” Nucleic Acids Res. 29:205-206 (2001).

By the phrase “humanized antibody” is meant an antibody that includes a humanized light chain and a humanized heavy chain immunoglobulin. See S. L. Morrison, supra; Oi et al., supra; Teng et al., supra; Kozbor et al., supra; Olsson et al., supra; and other references cited previously. Accordingly, a “humanized antibody fragment” means a part of that antibody, preferably a part that binds antigen specifically.

The H36 or cH36 antibody can be humanized by one or a combination of approaches as described, for example, in U.S. Pat. Nos. 5,766,886; 5,693,762; 5,985,279; 5,225,539; EP-A-0239400; 5,985,279 and 5,639,641, or as disclosed in the published U.S. application number 20030190705. See also E. Padlan Mol. Immunol. 28:489 (1991); Jones et al., Nature 321:522-525 (1986); Junghans et al., supra; and Roguska, et al. PNAS (USA) 91:969 (1994) for additional information on humanizing antibodies.

Particular humanized antibodies and fragments thereof for use with the present invention are disclosed below in the Examples section.

Preferred chimeric antibodies, humanized antibodies, as well as fragments thereof that specifically bind human TF. By the term, “specific binding” or a similar term is meant a molecule disclosed herein which binds another molecule, thereby forming a specific binding pair. However, the molecule does not recognize or bind to other molecules as determined by, e.g., Western blotting ELISA, RIA, mobility shift assay, enzyme-immunoassay, competitive assays, saturation assays or other protein binding assays know in the art. See generally, Sambrook et al. in Molecular Cloning: A Laboratory Manual (2nd ed. 1989); and Ausubel et al., supra; and Harlow and Lane in Antibodies: A Laboratory Manual (1988) Cold Spring Harbor, N.Y. for examples of methods for detecting specific binding between molecules.

Especially suitable chimeric antibodies, humanized antibodies and fragments for use with the invention will feature at least one of: 1) a dissociation constant (K_{d}) for the TF of less than about 0.5 nM; and 2) an affinity constant (Kₐ) for the TF of less than about 10×10¹⁰ M⁻¹. Methods of preforming such assays are known in the field and include Enzyme-Linked Immuno-Sorbent Assay (ELISA), Enzyme ImmunoAssay (EIA) radioimmunoas say (RIA) and BIAcore analysis.

Additionally suitable antibodies will increase survival time in what is sometimes referred to herein as a “standard in vivo septic shock assay”. Generally, such an assay involves administering gram-negative bacteria to monkeys to induce sepsis. See Taylor et al. J. Clin. Invest. 79:918 (1987). More specifically, a dose of E. coli (about 10¹⁰ CFU/kg) is freshly prepared and administered intravenously to a primate, e.g. baboon or rhesus monkey, in a 1-2 hour interval. A control group can receive a saline or PBS injection. The treatment group receives a bolus injection of at least 1 mg/kg antibody, preferably about 10 mg/kg prior to infusion of bacteria e.g., less than about 1 hour before. Control and treatment group monkeys are then monitored for about a week and checked for survival. See the Examples below for more specific information about the standard in vivo septic shock assay.

A preferred method of the invention employs a humanized antibody, chimeric antibody or fragment that increases monkey survival time (hours or days) by at least about 2-fold, preferably at least about 3 fold, more preferably at least about 5 fold to 10 fold or more as determined by the standard in vivo septic shock assay.

Additionally suitable antibodies for use with the invention can attenuate (reduce presence of at least one of interleukin-6 (IL-6) and interleukin-8 (IL-8) in the plasma of subject mammals after at least about 5 hours following administration of the antibody. Methods for detecting IL-6 and IL-8 and quantifying same from plasma are known and include immunological approaches such as RIA, EIA, ELISA and the like.

In one approach, sepsis is induced in a suitable primate such as a monkey and preferably a baboon along lines previously mentioned. Prior to inoculation of control baboon (with about 10¹⁰ CFU/kg E. coli or saline), the treatment baboons receive a bolus injection of at least 1 mg/kg antibody, preferably about 10 mg/kg prior to infusion of bacteria e.g., less than about 1 hour before injection of the inoculum. Baboon survival and IL-6 and IL-8 levels in plasma are monitored by standard procedures.

In another approach, a sepsis-like condition is induced in a suitable primate; such as baboon, using a model described as a “primed sepsis model” (see Welty-Wolf, K. et al. Am. J. Respir. Crit. Care Med. 164:1988 (2001) and described in further detail in Example 4).

Additionally preferred humanized antibodies, chimeric antibodies and fragments thereof exhibit a blood clotting time of between from about 50 to about 350 seconds as determined by a standard prothrombin (PT) time assay, particularly after about 5 minutes administration of the antibody or fragment to the mammal. An especially useful PT assay has been disclosed in the U.S. Pat. Nos. 5,986,065, 6,555,319 and PCT/US98/04644 (WO 98/40408), for example.

Further preferred antibodies for use in accord with the present invention inhibit platelet deposition by at least about 50% as determined by a standard platelet deposition assay. Methods for performing the assay have been disclosed, e.g., in the pending U.S. patent application Ser. No. 10/310,113.

Still further preferred antibodies inhibit collagen-induced arthritis in an experimental mouse model. See Example 5.

“Antibody”, “antibody for use with the invention” and like phrases refer to whole immunoglobulin as well as immunologically active fragments which bind a desired antigen. The immunoglobulins and immunologically active (antigen-binding) fragments thereof include an epitope-binding site (i.e., a site or epitope capable of being specifically bound by an antibody recognizing antigen). Exemplary antibody fragments include, for example, Fab, F(v), Fab′, F(ab′)₂ fragments, “half molecules” derived by reducing the disulfide bonds of immunoglobulins, single chain immunoglobulins, or other suitable antigen binding fragments (see e.g., Bird et al., Science, 242:423-426 (1988); Huston et al., PNAS, (USA), 85:5879 (1988); Webber et al., Mol. Immunol., 32:249 (1995)).

Particular chimeric or humanized antibodies of the present invention can be polyclonal or monoclonal, as needed, and may have, without limitation, an IgG1, IgG2, IgG3 or IgG4 isotype or IgA, IgD, IgE, IgM. An especially preferred antibody for use with the invention has or can be manipulated to have an IgG1 (called “hOAT”) or IgG4 (called “hFAT”) isotype. Such antibodies can be polyclonal or monoclonal as needed to achieve the objectives of a particular invention embodiment. In some instances, single-chain antibodies such as a humanized single-chain will be preferred.

Practice of the invention is applicable to a wide spectrum of mammals. Preferably, the mammal is a primate such as a monkey, chimpanzee, or a baboon. More preferably, the primate is a human patient in need of methods disclosed herein i.e., one that has or is suspected of having sepsis or a sepsis-related condition or an inflammatory disorder or disease.

As discussed above, antibodies of the invention and suitable fragments thereof can be administered to a mammal, preferably a primate such as a human, to prevent or reduce sepsis and related complications. According to one embodiment, such antibodies are used with one or more pharmaceutically acceptable non-toxic carriers such as sterile water or saline, glycols such as polyethylene glycol, oils of vegetable origin, and the like. In particular, biocompatible, biodegradable lactide polymer, lactide glycolide copolymer or polyoxyethylene, polyoxypropylene copolymers may be useful excipients. Other potentially useful administration systems include ethylene vinyl acetate copolymer particles, osmotic pumps, and implantable infusion systems and liposomes. If needed, one or more suitable antibodies or fragments will be in the form of a solution or suspension (or a lyophilized form that can be reconstituted to a solution or suspension), and will preferably include approximately 0.01% to 10% (w/w) of the antibody of the present invention, preferably approximately 0.01% to 5% (w/w) of the antibody.

As discussed, the antibody or fragment can be administered according to the invention as the sole active agent or in combination with other known anti-sepsis therapies. For instance, such antibodies or fragments can be administered to a human patient before, during, or after intervention with one or more appropriate antibotics, typically a broad spectrum intravenous antibiotic therapy. Preferred antibiotics include those known to have broad spectrum anti-microbial activity, covering gram-positive, gram-negative, and anaerobic bacteria. Thus in one embodiment, such antibiotics are given during or after administration of the antibodies and fragments disclosed herein, preferably parenterally in doses adequate to achieve bactericidal serum levels. After stabilizing the patient, various recognized supportive therapies can be used to assist recovery such as administration of oxygen, intravenous fluids, and medications that increase blood pressure. Dialysis may be necessary in the event of kidney failure, and mechanical ventilation is often required if respiratory failure occurs.

Persons “at risk” for developing sepsis and related conditions include, but are not limited to, the very old and very young individuals. Also at risk are those with challenged immune systems such as patients infected with a DNA or RNA virus such as HIV or herpes. Nearly any bacterial organism can cause sepsis. Certain fungi and (rarely) viruses may faciliate sepsis and related conditions. Generally, toxins released by the bacteria or fungus may cause direct organ (e.g., lung, kidney) or tissue damage, and may lead to low blood pressure and/or poor organ function. These toxins also produce a vigorous inflammatory response from the body which contributes to septic shock.

Additional risk factors include underlying illnesses, such as diabetes; hematologic cancers (lymphoma or leukemia); and other malignancies and diseases of the genitourinary system, liver or biliary system, and intestinal system. Other risk factors are recent infection, prolonged antibiotic therapy, and having been exposed to a recent invasive surgical or medical procedure. Symptoms of sepsis and related conditions are known in the field and include, but are not limited to, fever, chills, lightheadedness, shortness of breath, palpitations, cool and/or pale extremities, fever, agitation, lethargy, and confusion.

Success of the invention in the prevention or treatment of sepsis and related conditions can be evaluated by the caregiver using one or a combination of approaches. Typically, a reduction or elimination of one or more of the foregoing symptoms can be taken as indicative that the sepsis or related condition has been addressed. Preferably, patients receiving such treatment will survive at least about 2 fold longer than patients who do not receive such intervention.

As discussed, the invention provides a method for reducing cytokine production in a mammal e.g., by administering to the mammal a therapeutically effective amount of at least one humanized antibody, chimeric antibody, or fragment thereof that binds specifically to tissue factor (TF) to form a complex. Preferably, Factor X or Factor IX binding to the complex is inhibited and the administration is sufficient to reduce the cytokine production in the mammal. Suitable humanized antibodies, chimeric antibodies and fragments thereof are disclosed herein. Acceptable methods for monitoring cytokine production in a mammal are known and include specific methods outlined in the Examples section.

Therapeutic antibodies and fragments in accord with the invention can be used in parenteral or intravenous administration, particularly in the form of liquid solutions. Such compositions may be conveniently administered in unit dose and may be prepared in accordance with methods known in the pharmaceutical art. See Remington's Pharmaceutical Sciences, (Mack Publishing Co., Easton Pa., (1980)). By the term “unit dose” is meant a therapeutic composition of the present invention employed in a physically discrete unit suitable as unitary dosages for a primate such as a human, each unit containing a pre-determined quantity of active material calculated to produce the desired therapeutic effect in association with the required diluent or carrier. The unit dose will depend on a variety of factors including the type and severity of sepsis to be treated, general health of the individual, medical history, and the like. Precise amounts of the antibody to be administered typically will be guided by judgment of the practitioner, however, the unit dose will generally depend on the route of administration and be in the range of 10 ng/kg body weight to 50 mg/kg body weight per day, more typically in the range of 100 ng/kg body weight to about 10 mg/kg body weight per day. Suitable regimens for initial administration in booster shots are also variable but are typified by an initial administration followed by repeated doses at one or more hour intervals by a subsequent injection or other administration. Alternatively, continuous or intermittent intravenous infusions may be made sufficient to maintain concentrations of at least from about 10 nanomolar to 10 micromolar of the antibody (or suitable fragment) in the blood.

Antibodies and fragments for use with the invention are preferably substantially pure when used in the disclosed methods and assays. References to an antibody being “substantially pure” mean an antibody or protein that has been separated from components which naturally accompany it. For example, by using standard immunoaffinity or protein A affinity purification techniques, an antibody of the invention can be purified from hybridoma or cell culture medium by using native TF as an antigen or protein A resin. Similarly, native TF can be obtained in substantially pure form by using an antibody of the invention with standard immunoaffinity purification techniques. Particularly, an antibody or protein is substantially pure when at least 50% of the total protein (weight % of total protein in a given sample) is an antibody or protein of the invention. Preferably the antibody or protein is at least 60 weight % of the total protein, more preferably at least 75 weight %, even more preferably at least 90 weight %, and most preferably at least 98 weight % of the total material. Purity can be readily assayed by known methods such as SDS polyacrylamide gel electrophoresis (PAGE), column chromatography (e.g., affinity chromatography, size exclusion chromatography), mass spectroscopy or HPLC analysis. Preferably, the antibodies and fragments will be used in a sterile format.

The molecular weight of the antibodies of the invention will vary depending on several factors such as the intended use and whether the antibody includes a conjugated or recombinantly fused toxin, pharmaceutical, radioisotope or detectable label or the like. Also the molecular weight will vary depending on nature and extent of post-translational modifications if any (such as glycosylation) to the antibody. The modifications are a function of the host used for expression with E. coli producing non-glycosylated antibodies and eucaryotic hosts, such as mammalian cells or plants, producing glycosylated antibodies. In general, an antibody of the invention will have a molecular weight of between approximately 20 to 150 kDa. Such molecular weights can be readily determined by molecular sizing methods such as SDS-PAGE followed by protein staining or Western blot analysis.

It will be apparent that the foregoing method for making the humanized cH36 antibody can be readily adapted to make other humanized antibodies and antigen binding fragments according to the invention.

### A. Preparation of Humanized Anti-Tissue Factor Binding Antibody

Preparation and use of a humanized anti-tissue factor binding antibody is described. See also Examples 1-3 below.

Briefly, preferred antibodies bind human tissue factor to form a binding complex. The tissue factor may be naturally occurring or recombinant human (rhTF). Preferably, factor X or factor IX binding to the complex is inhibited. In a preferred invention embodiment, the humanized antibody has an apparent affinity constant (K_{A}, M⁻¹) for the hTF of less than about 1 nM, preferably less than about 0.5 nM, more preferably between from about 0.01 nM to about 0.4 nM. See Examples 1-3, below for more information about determining affinity constants for the humanized antibodies. By “specific binding” is meant that the humanized antibodies form a detectable binding complex with the TF (or rhTF) and no other antigen as determined by standard immunological techniques such as RIA, Western blot or ELISA.

More preferred humanized anti-TF binding antibodies made in accord with this invention exhibit an apparent affinity constant (K_{A}, M⁻¹) for native human TF of at least about 1×10⁸M⁻¹ as determined by surface plasmon analysis (particularly, BIACore analysis in accordance with the procedures of Example 3 which follows), more preferably at least about 5×10⁸ M⁻¹ as determined by surface plasmon analysis, still more preferably an apparent affinity constant (K_{A}, M⁻¹) for native human TF of at least about 3×10⁹ M⁻¹ as determined by surface plasmon resonance analysis. Such substantial binding affinity of antibodies of the invention contrast sharply from much lower binding affinities of previously reported antibodies.

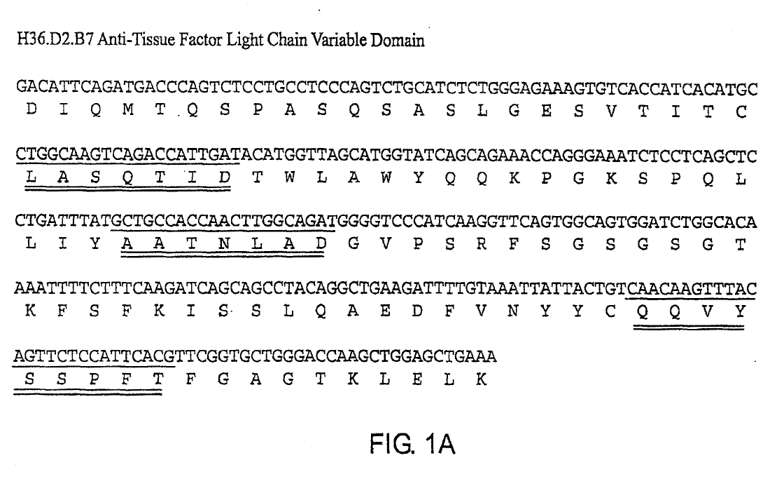

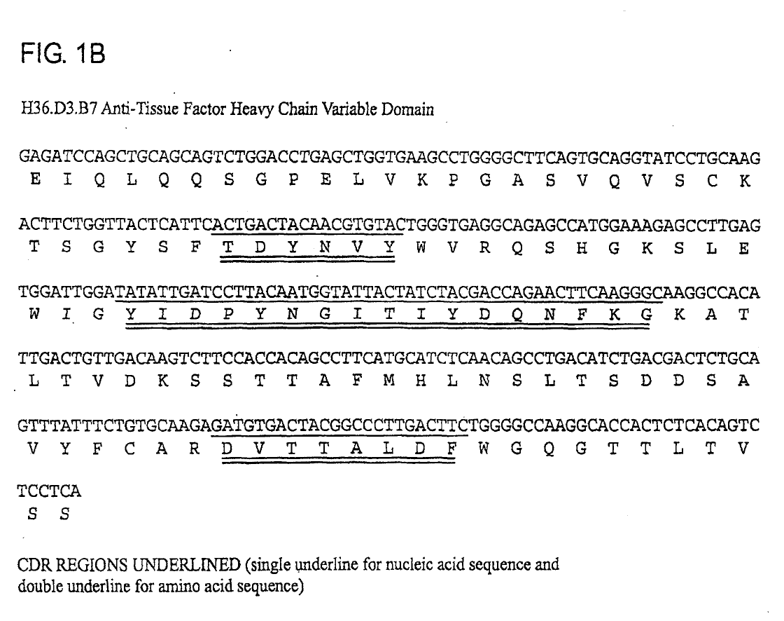

The nucleic acid (SEQ ID NOS. 1 and 3) and amino acid (SEQ ID NOS. 2 and 4) sequences of a particular tissue factor binding antibody that has been humanized by the present methods i.e. H36.D2.B7. See FIGS. 1A and 1B of the drawings and the PCT application WO 98/40408, for instance. SEQ ID NOS. 1 and 2 are the nucleic acid and amino acid respectively of the light chain variable domain, and SEQ ID NOS. 3 and 4 are the nucleic acid and amino acid respectively of the heavy chain variable domain, with hypervariable regions (CDRs or Complementarity Determining Regions) underlined in all of those sequences.

Additional tissue factor binding humanized antibodies of the invention will have substantial amino acid sequence identity to either one or both of the light chain or heavy sequences shown in FIGS. 1A and 1B. More particularly, such antibodies include those that have at least about 70 percent homology (amino acid sequence identity) to SEQ ID NOS. 2 and/or 4, more preferably about 80 percent or more homology to SEQ ID NOS. 2 and/or 4, still more preferably about 85, 90 or 95 percent or more homology to SEQ ID NOS. 2 and/or 4.

More particular tissue factor binding humanized antibodies of the invention will have high amino acid sequence identity to hypervariable regions (shown with double underlining in FIGS. 1A and 1B) of SEQ ID NOS. 2 and 4). Specific antibodies will have one, two or three hypervariable regions of a light chain variable domain that has high sequence identity (at least 90% or 95% amino acid sequence identity) to or be the same as one, two or three of the corresponding hypervariable regions of the light chain variable domain of H36.D2.B7 (those hypervariable regions shown with underlining in FIG. 1A and are the following:

(SEQ ID NO. 5)
1) LASQTID;
(SEQ ID NO. 6)
2) AATNLAD;
and
(SEQ ID NO. 7)
3) QQVYSSPFT.

Additionally specific antibodies that have been humanized by the methods described herein and bind tissue factor will have one, two or three hypervariable regions of a heavy chain variable domain that have high sequence identity (at least 90% or 95% amino acid sequence identity) to or be the same as one, two or three of the corresponding hypervariable regions of the heavy chain variable domain of H36.D2.B7 (those hypervariable regions shown with underlining in FIG. 1B and are the following:

(SEQ ID NO. 8)
1) TDYNVY;
(SEQ ID NO. 9)
2) YIDPYNGITIYDQNFKG;
and
(SEQ ID NO. 10)
3) DVTTALDF.

Certain nucleic acids encoding some or all of the antibodies or fragments disclosed herein will preferably have a length sufficient (preferably at least about 100, 200 or 250 base pairs) to bind to the sequence of SEQ ID NO. 1 and/or SEQ ID NO. 3 under the following moderately stringent conditions (referred to herein as “normal stringency” conditions): use of a hybridization buffer comprising 20% formamide in 0.9M saline/0.12M sodium citrate (6×SSC) buffer at a temperature of 37° C. and remaining bound when subject to washing once with that 2×SSC buffer at 37° C.

More specifically, certain of the nucleic acids (preferably at least about 100, 200 or 250 base pairs) will bind to the sequence of SEQ ID NO. 1 and/or SEQ ID NO. 3 under the following highly stringent conditions (referred to herein as “high stringency” conditions): use of a hybridization buffer comprising 20% formamide in 0.9M saline/0.12M sodium citrate (6×SSC) buffer at a temperature of 42° C. and remaining bound when subject to washing twice with that 1×SSC buffer at 42° C.

Suitable nucleic acids preferably comprise at least 20 base pairs, more preferably at least about 50 base pairs, and still more preferably a nucleic acid of the invention comprises at least about 100, 200, 250 or 300 base pairs.

Generally preferred nucleic acids of the invention will express an antibody of the invention that exhibits the preferred binding affinities and other properties as disclosed herein. See also the U.S. Pat. No. 5,986,065 and PCT/US98/04644 (WO 98/40408) for more information.

Other appropriate nucleic acids will have substantial sequence identity to either one or both of the light chain or heavy sequences shown in FIGS. 1A and 1B. More particularly, preferred nucleic acids will comprise a sequence that has at least about 70 percent homology (nucleotide sequence identity) to SEQ ID NOS. 1 and/or 3, more preferably about 80 percent or more homology to SEQ ID NOS. 1 and/or 3, still more preferably about 85, 90 or 95 percent or more homology to SEQ ID NOS. 1 and/or 3.

Additionally specific nucleic acid sequences will have high sequence identity to hypervariable regions (shown with underlining in FIGS. 1A and 1B) of SEQ ID NOS. 1 and 3). Such nucleic acids include ‘those that code for an antibody light chain variable domain and have one, two or three sequences that code for hypervariable regions and have high sequence identity (at least 90% or 95% nucleotide sequence identity) to or be the same as one, two or three of the sequences coding for corresponding hypervariable regions of H36.D2.B7 (those hypervariable regions shown with underlining in FIG. 1A and are the following:

(SEQ ID NO: 11)
1) CTGGCAAGTCAGACCATTGAT;
(SEQ ID NO: 12)
2) GCTGCCACCAACTTGGCAGAT;
and
(SEQ ID NO: 13)
3) CAACAAGTTTACAGTTCTCCATTCACGT.

More specific nucleic acids also code for an antibody heavy chain variable domain and have one, two or three sequences that code for hypervariable regions and have high sequence identity (at least 90% or 95% sequence identity) to or be the same as one, two or three of the sequences coding for corresponding hypervariable regions of H36.D2.B7 (those hypervariable regions shown with underlining in FIG. 1B and are the following:

(SEQ ID NO. 14)
1) ACTGACTACAACGTGTAC;
(SEQ ID NO. 15)
2) TATATTGATCCTTACAATGGTATTACTATCTACGACCAGAACTTCA
AGGGC;
and
(SEQ ID NO. 16)
3) GATGTGACTACGGCCCTTGACTTC.

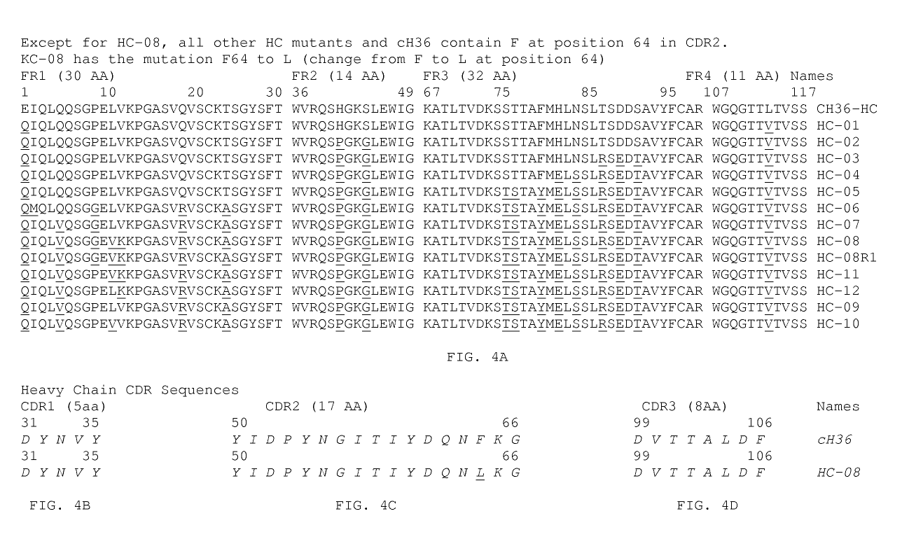

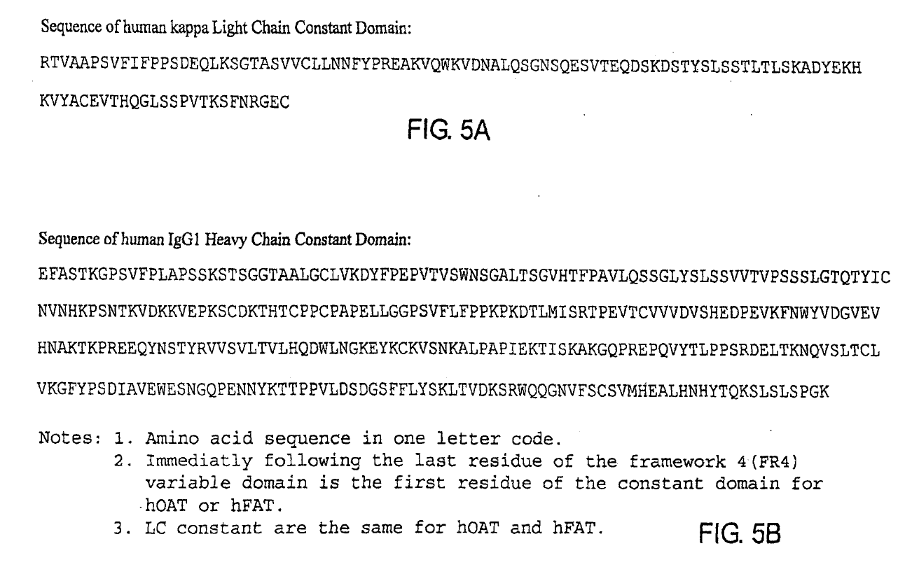

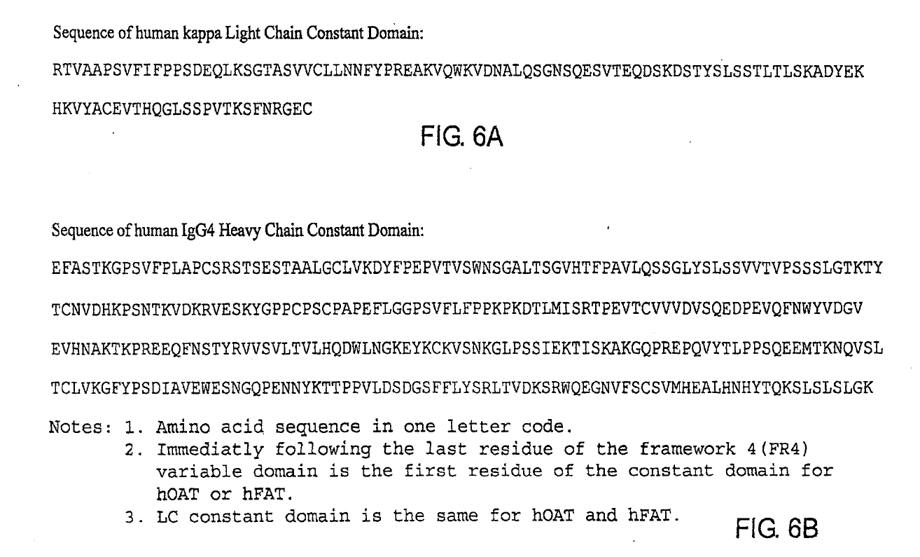

More specific humanized antibodies for use with the methods of this invention that bind TF are those in which each of framework regions (FRs) 1, 2, 3 and 4 has at least about 90% amino acid sequence identity, preferably at least about 95% or greater identity to the light chain FR sequences shown in FIG. 3A (SEQ ID NO. ______), preferably, the sequence shown as “LC-09” in FIG. 3A. Additionally specific humanized antibodies include a light chain constant domain having at least about 90% amino acid sequence identity, preferably at least about 95% sequence identity or greater to the sequence shown in FIG. 5A (SEQ ID NO. ______) or FIG. 6A (SEQ ID NO. ______).

Further specific humanized antibodies are those in which each of framework regions (FRs) 1, 2, 3 and 4 has at least about 90% amino acid sequence identity, preferably about 95% identity or greater to the heavy chain sequences shown in FIG. 4A (SEQ ID NO. ______, preferably, the sequence shown as “HC-08” in FIG. 4A. Additional humanized antibodies have a heavy chain constant domain with at least about 90% amino acid sequence identity, preferably at least about 95% identity or greater, to sequence shown in FIG. 5B (SEQ ID NO. ______ or FIG. 6B (SEQ ID NO. ______).

In certain embodiments, the humanized antibody will have an IgG1 (hOAT) or IgG4 (hFAT) isotype as disclosed in the published U.S. application number 20030190705.

Also provided by the present invention are functional fragments of the humanized antibodies disclosed herein. Examples of such fragments include, but are not limited to, those that bind TF with an affinity constant (Kd) of less than about 1 nM, preferably less than about 0.5 nM, more preferably between from about 0.01 nM to about 0.4 nM. Specifically preferred are antigen binding Fab, Fab′, and F(ab)₂ fragments.

As discussed, the invention features humanized antibodies that include at least one murine complementarity determining region (CDR), e.g., CDR1, CDR2, CDR3. In one invention embodiment, the antibodies bind specifically to human tissue factor (TF) to form a complex. Typically, the factor X or factor IX binding to TF or TF-FVIIa and activation by TF-FVIIa thereto is inhibited. As mentioned above, preferred CDRs (light and heavy chain) are from a rodent source, typically the mouse.

In one embodiment of the humanized antibodies of the invention, the antibodies further include at least one human framework region (FR) subset. Preferably, all the FRs (light and heavy chains) are human.

In a more particular embodiment, the first CDR (CDR1) of the heavy chain hypervariable region that binds human TF is at least 90% identical to the CDR1 amino acid sequence shown in FIG. 4B (SEQ ID NO. ______), preferably at least about 95% identical or greater to that sequence. Typically, the second CDR (CDR2) of the heavy chain hypervariable region is at least 90% identical to the CDR2 amino acid sequence shown in FIG. 4C (SEQ ID NO. ______, preferably at least about 95% identical or greater. Preferably also, the third CDR (CDR3) of the heavy chain hypervariable region is at least 90% identical to the CDR3 sequence shown in FIG. 4D (SEQ ID NO. ______, more preferably about 95% identical or greater to that sequence.

In another invention embodiment, the first CDR (CDR1) of the light chain hypervariable region that binds human TF is at least 90% identical to the CDR1 amino acid sequence shown in FIG. 3B (SEQ ID NO. ______), preferably at least about 95% identical or greater. Typically, the second CDR (CDR2) of the light chain hypervariable region is at least 90% identical to the CDR2 amino acid sequence shown in FIG. 3C (SEQ ID NO. ______), preferably about 95% identical or greater. Preferably, the third CDR (CDR3) of the light chain hypervariable region is at least 90% identical to the CDR3 amino acid sequence shown in FIG. 3D (SEQ ID NO. ______), more preferably about 95% identical or greater to that sequence.

Additional humanized antibodies suitable for use with the present methods include a first framework region (FR1) of the heavy chain hypervariable region that binds human TF which FR1 is at least 90% identical to the amino acid sequence shown in FIG. 4A (SEQ ID NO. ______) as “FR1 HC-08”, preferably about 95% identical or greater to that sequence. In one embodiment, the FR1 comprises at least one of the following amino acid changes: E1 to Q; Q5 to V; P9 to G; L11 to V; V12 to K; Q19 to R; and T24 to A. Preferably, the FR1 includes two, three, four, five, or six of those changes with all of those amino acid changes being preferred for many applications.

Further humanized antibodies suitably bind human TF and include a second framework region (FR2) of the heavy chain hypervariable region which FR2 is at least 90% identical to the sequence shown in FIG. 4A (SEQ ID NO. ______) as “FR2 HC-08”, preferably about 95% identical or greater to that sequence. In one embodiment, the FR2 at least one of the following amino acid changes: H41 to P and S44 to G. A preferred FR2 includes both of those amino acid changes.

The invention also features use of humanized antibodies that bind human TF in which a third framework region (FR3) of the heavy chain hypervariable region is at least 90% identical to the sequence shown in FIG. 4A (SEQ ID NO. ______) as “FR3 HC-08”, preferably about 95% identical or greater to that sequence. In one embodiment, the FR3 includes at least one of the following amino acid changes: S76 to T; T77 to S; F80 to Y; H82 to E; N84 to S; T87 to R; D89 to E and S91 to T. A preferred FR3 includes two, three, four, five or six of those amino acid changes with all seven of those amino acid changes being generally preferred.

Also featured is use of humanized antibodies that suitably bind human TF and in which the fourth framework region (FR4) of the heavy chain hypervariable region is at least 90% identical to the amino acid sequence shown in FIG. 4A (SEQ ID NO. ______) as “FR4 HC-08”, preferably at least about 95% identical or greater to that sequence. Preferably, the FR4 includes the following amino acid change: L113 to V.

Additional humanized antibodies bind human TF and also feature a first framework region (FR1) of the light chain hypervariable region which is at least about 90% identical to the amino acid sequence shown in FIG. 3A (SEQ ID NO. ______) as “FR1 LC-09”, preferably at least about 95% identical or greater to that sequence. In one embodiment, the FR1 comprises at least one of the following amino acid changes: Q11 to L; L15 to V; E17 to D; and S18 to R. A preferred FR1 includes two or three of such amino acid changes with all four amino acid changes being generally preferred.

The present invention also features use of humanized antibodies. that bind human TF and in which a second framework region (FR2) of the light chain hypervariable region is at least about 90% identical to the amino acid sequence shown in FIG. 3A (SEQ ID NO. ______) as “FR2 LC-09”, preferably at least about 95% identical or greater to that sequence. A preferred FR2 has the following amino acid change: Q37 to L.

Also encompassed by the invention is use of particular humanized antibodies that bind human TF in which a third framework region (FR3) of the light chain hypervariable region is at least about 90% identical to the amino acid sequence shown in FIG. 3A (SEQ ID NO. ______) as “FR3 LC-09”, preferably at least about 95% identical or greater to that sequence. In one embodiment, the FR3 has at least one of the following amino acid changes: K70 to D, K74 to T, A80 to P, V84 to A, and N85 to T. Preferably, the FR3 has two, three, or four of such amino acid changes with all five of the changes being generally preferred.

Additional humanized antibodies appropriate for use with the methods disclosed herein bind TF and include a fourth framework region (FR4) of the light chain hypervariable region which FR4 is at least about 90% identical to the sequence shown in FIG. 3A (SEQ ID NO. ______) as “FR4 LC-09”, preferably at least about 95% identical or greater to that sequence. In one embodiment, the FR4 includes at least one and preferably all of the following amino acid changes: A100 to Q and L106 to I.

The invention also features a human TF binding fragment of the foregoing humanized antibodies. Examples of such fragments include Fab, Fab′, and F(ab)₂. See the published U.S. application number 20030190705 and references cited therein for additionally preferred humanized anti-TF antibodies made in accord with this invention.

The following three nucleic acid vectors pSUN36 (humanized anti-TF antibody Ig G1-HC expression vector), pSUN37 (humanized anti-TF antibody Ig G4-HC expression vector), and pSUN38 (humanized anti-TF antibody LC expression vector) have been deposited pursuant to the Budapest Treaty with the American Type Culture Collection (ATCC) at 10801 University Boulevard, Manassas Va. 20110-2209. The vectors were assigned the following Accession Numbers: PTA-3727 (pSUN36); PTA-3728 (pSUN37); and PTA-3729 (pSUN38).

Suitable expression and purification strategies for making and using the humanized anti-TF antibodies of this invention have been disclosed in the published U.S. application number 20030190705, for instance.

As discussed, the invention also provides useful kits for performing one or more of the methods provided herein. In one embodiment, the kit includes at least one humanized antibody, chimeric antibody, or fragment thereof that binds specifically to human tissue factor (TF) to form a complex, wherein factor X or factor IX binding to the complex is inhibited. Typically also, the humanized antibody, chimeric antibody, or fragment thereof is provided in a pharmaceutically acceptable vehicle such as saline, water or buffer. The kit may include a pharmaceutically acceptable vehicle for dissolving the humanized antibody, chimeric antibody or fragment prior to use.

The following non-limiting examples are illustrative of the invention. In the following examples and elsewhere the method of the invention is applied to the humanization of the murine anti-tissue factor antibody H36. See the U.S. Pat. Nos. 5,986,065 and 6,555,319; U.S. Patent Application Publication No. 20030082636; WO 03/037911 and WO 98/40408, and the published U.S. patent application number 20030190705 and references cited therein as well as the discussion above. Sometimes, the Fab fragment of H36 or cH36 will be referred to as “H36-Fab” and “cH36-Fab”, respectively, for the sake of convenience.

All documents mentioned herein are fully incorporated by reference in their entirety.

### Example 1

### Humanization of Anti-Tissue Factor Antibody

The description of how to make and use a particular murine antibody called H36.D2 (sometimes also called H36 as discussed above) is described in U.S. Pat. Nos. 5,986,065 and 6,555,319. The present example shows how to make and use a humanized version of that antibody. A humanized H36 antibody has a variety of uses including helping to minimize potential for human anti-mouse antibody (HAMA) immunological responses. These and other undesired responses pose problems for use of the H36 antibody in human therapeutic applications.

A. Preparation of Chimeric Anti-Tissue Factor Antibody (cH36)

The H36 antibody described previously is an IgG2a murine antibody. H36 was first converted to a mouse-human chimeric antibody for clinical development. To do this, the heavy and light chain genes for H36 were cloned (see U.S. Pat. No. 5,986,065). The heavy chain variable domain was fused to a human IgG4 constant (Fe) domain and the light chain variable domain was fused to a human kappa light chain constant domain. The resulting IgG4κ chimeric antibody was designated cH36 (and is also referred to as Sunol-cH36). For multiple uses of H36 or cH36 in patients with chronic diseases, a fully humanized cH36 is preferred so that it will decease or eliminate any human anti-chimeric antibody (HACA) immunological response. The humanization of cH36 is described below.

B. Humanization Strategy for cH36 Antibody

Humanization of the chimeric anti-tissue factor antibody cH36 was achieved by using a “FR best-fit” method of the invention. This method takes full advantage of the fact that a great number of human IgGs with known amino acid sequences or sequences of human IgG fragments are available in the public database. The sequences of the individual framework regions of the mouse heavy and light variable domains in cH36 are compared with the sequences respective heavy or light chain variable domains or human frameworks (or fragments thereof) in the Kabat database (see e.g., Kabat et al. in Sequences of Proteins of Immunological Interest Fifth Edition, U.S. Dept. of Health and Human Services, U.S. Government Printing Office (1991) NIH Publication No. 91-3242 or http://immuno.bme.nwu.edu). The following criteria were used to select the desired human IgG framework region subsets for humanization: (1) The number of mismatched amino acids was kept as low as possible. (2) Amino acids inside the “vernier” zone (amino acids in this zone may adjust CDR structure and fine-tune the fit to antigen, see Foote, J. and Winter, G., J. of Mol. Bio. 224(2):487-499 [1992]) were left unchanged. (3) Conservative amino acid substitutions were favored when evaluating similar candidates. The matching program used for this comparison can be found in Kabat database. See Johnson G, Wu T. “Kabat database and its application: Future directions.” Nucleic Acids Res. 29:205-206 (2001). The program finds and aligns regions of homologies between the mouse sequences and human sequences in the Kabat's database. By using this unique FR best-fit method, it is anticipated that the humanized LC or HC variable domains of the target IgG may have all the four FRs derived from as few as one human IgG molecule or to as many as four different human IgG molecules.

### B(i). Selection of Human Igg Kappa Light Chain Variable Domain Framework Regions

The amino acid sequence in each of the framework regions of cH36 LC was compared with the amino acid sequence in the FRs in human IgG kappa light chain variable domain in Kabat Database. The best-fit FR was selected based on the three criteria described above.

The amino acid sequence of human IgG kappa light chain variable domain with a Kabat Database ID No. 005191 was selected for humanization of cH36 LC FR1. The amino acid sequence of human IgG kappa light chain variable domain with a Kabat Database ID No. 019308 was selected for humanization of cH36 LC FR2. The following mutations were made in cH36 LC FR1 to match the amino acid sequence of a human IgG kappa light chain variable domain with a Kabat Database ID No. 005191: Q11→L, L15→V, E17→D, S18→R. One mutation Q37→L was made cH36 LC FR2 to match the amino acid sequence of a human IgG kappa light chain variable domain with a Kabat Database ID No. 019308 (see Table 1A for sequence information).

The amino acid sequence of a human IgG kappa light chain variable domain with a Kabat Database ID No. 038233 was selected for humanization of cH36 LC FR3. The amino acid sequence of a human IgG kappa light chain variable domain with a Kabat Database ID No. 004733 was selected for humanization of cH36 LC FR4. The following mutations were made in cH36 LC FR3 to match the amino acid sequence of a human IgG kappa light chain variable region with a Kabat Database ID No. 038233: K70→D, K74→T, A80→P, V84→A, N85→T. Two mutations A100→Q and L106→I were made cH36 LC FR4 to match the amino acid sequence of a human IgG kappa light chain variable domain with a Kabat Database ID No. 004733 (see Table 1B for sequence information).

### B(ii). Selection of Human IgG Heavy Chain Variable Domain Framework Regions

The amino acid sequence in each of the framework regions of cH36 HC was compared with the amino acid sequence in the FRs in human IgG heavy chain variable domain in Kabat Database. The best-fit FR was selected based on the three criteria described above.

The amino acid sequence of a human IgG heavy chain variable domain with a Kabat Database ID No. 000042 was selected for humanization of cH36 HC FR1. The amino acid sequence of a human IgG heavy chain variable domain with a Kabat Database ID No. 023960 was selected for humanization of cH36 HC FR2. The following mutations were made in cH36 HC FR1 to match the amino acid sequence of a human IgG heavy chain variable domain with a Kabat Database ID No. 000042: E1→Q, Q5→V, P9→G, L11→V, V12→K, Q19→R, T24→A. Two mutations H41→P and S44→G were made cH36 HC FR2 to match the amino acid sequence of a human IgG heavy chain variable domain with a Kabat Database ID No. 023960 (see Table 2A for sequence information).

The amino acid sequence of a human IgG heavy chain variable domain with a Kabat Database ID No. 037010 was selected for humanization of cH36 HC FR3. The amino acid sequence of a human IgG heavy chain variable domain with a Kabat Database ID No. 000049 was selected for humanization of cH36 HC FR4. The following mutations were made in cH36 HC FR3 to match the amino acid sequence of a human IgG heavy chain variable domain with a Kabat Database ID No. 037010: S76→T, T77→S, F80→Y, H82→E, N84→S, T87→R, D89→E, S91→T. One mutations L113→V was made cH36 HC FR2 to match the amino acid sequence of a human IgG heavy chain variable domain with a Kabat Database ID No. 000049 (see Table 2B for sequence information).

TABLE 1
Comparison of cH36 and Human Light Chain (LC) FR Sequences
Table 1A

| Names | LC-FR1 (23 aa) | LC-FR2 (15 aa) |
|---|---|---|
| | 1 10 20 | 35 49 |
| cH36-LC | DIQMTQSPASQSASLGESVTITC | WYQQKPGKSPQLLIY |
| Human-LC | L V DR | L |
| | 005191 | 019308 |

Table 1B

| Names | LC-FR3 (32 aa) | LC-FR4 (10 aa) |
|---|---|---|
| | 57 60 70 80 88 | 98 |
| 107 | | |
| cH36-LC | GVPSRFSGSGSGTKFSFKISSLQAEDFVNYYC | FGAGTKLELK |
| Human-LC | D T P AT | Q I |
| | 038233 | 004733 |

TABLE 2
Comparison of cH36 and Human Heavy Chain (HC) FR Sequences
Table 2A

| Names | HC-FR1 (30 aa) | HC-FR2 (14 aa) |
|---|---|---|
| | 1 10 20 30 | 36 |
| cH36-HC | EIQLQQSGPELVKPGASVQVSCKTSGYSFT | |
| WVRQSHGKSLEWIG | | |
| Human-HC | Q V G VK R A | P G |
| | 000042 | 023960 |

Table 2B

| Names | HC-FR3 (32 aa) | HC-FR4 (11 aa) |
|---|---|---|
| | 67 75 85 95 | 107 117 |
| cH36-HC | KATLTVDKSSTTAFMHLNSLTSDDSAVYFCAR | WGQGTTLTVSS |
| Human-HC | TS Y E S R E T | V |
| | 037010 | 00049 |

Once the decisions on the desired human framework regions were made, the following three techniques were used to achieve the desired amino acid substitutions in both the light and heavy chains: (1) Regular PCR was used for cloning, to introduce cloning or diagnostic restriction endonuclease sites, and to change amino acid residues located at the ends of the variable domains. (2) PCR-based mutagenesis was used to change multiple amino acid residues at a time, especially when these residues were in the middle of the variable domains. (3) Site-directed mutagenesis was used to introduce one or two amino acid substitutions at a time. Site-directed mutagenesis was done following the protocol described in Stratagene's “QuickChange Site-Directed Mutagenesis Kit” (Catalog #200518).

After each step, the partially humanized clones were sequenced and some of these variable domains were later cloned into expression vectors. The plasmid tKMC180 was used to express LC mutants, and the pJRS 355 or pLAM 356 vector was used to express HC mutants as IgG1 or IgG4, respectively. Some of these clones were then combined and expressed transiently in COS cells to determine the expression levels by ELISA.

The final fully humanized forms of the anti-TF heavy and light variable domains were cloned into what is sometimes referred to herein as a “mega vector” and transfected into CHO and NSO cells for IgG expression. Stable cell lines were then used to produce amounts of humanized anti-TF sufficient for analysis. The resulting humanized versions are 100% human in origin (when the CDR sequences are not considered). The humanized IgG4 kappa version is designated hFAT (humanized IgG Four Anti-Tissue Factor antibody) and the IgG1 kappa version is designated hOAT (humanized IgG One Anti-Tissue Factor antibody). These fully humanized versions of cH36 are intended for treating chronic indications, such as thrombosis, cancer and inflammatory diseases.

### C. Generation of Humanized Anti-TF Antibody Heavy Chain

1. PCR amplification and cloning into pGem T-easy of anti-TF mAb cH36 heavy chain (HC) variable domain were performed using plasmid pJAIgG4TF.A8 (an expression vector for chimeric H36) as template and primers TFHC1s2 and TFHC1as2. Primer TFHC1s2 introduced a BsiW1 site upstream of the initiation codon and also an amino acid change E1 to Q in framework (FR) 1. Primer TFHC1 as introduced an amino acid change L113 to V in FR4. This step resulted in the construct HC01.
2. PCR-based mutagenesis using the previous construct (HC01) and the following four primers generated construct HC02. Upstream PCR used primers TFHC1s2 and TFHC7 as. Downstream PCR used primers TFHC7s and TFHC1as2. PCR using upstream and downstream PCR products as templates and with primers TFHC1s2 and TFHC1as2 yielded HC02. The use of primers TFHC7s and TFHC7 as introduced two amino acid changes in FR2: H41 to P and S44 to G.
3. PCR-based mutagenesis using HC02 as template and the following four primers generated construct HC03. Upstream PCR used primers TFHC1s2 and TFHC5 as2. Downstream PCR used primers TFHC5s and TFHC1as2. PCR using upstream and downstream PCR products as templates and with primers TFHC1s2 and TFHC1as2 yielded HC03. The use of primers TFHC5s and TFHC5 as2 introduced three amino acid changes in FR3: T87 to R, D89 to E, and S91 to T. A Bgl II site was also introduced at position 87.
4. PCR amplification was performed using primers TFHC2s and TFHC3 as and HC03 in pGem as template. TFHC2s sits upstream of the cloning site in pGem. TFHC3 as sits in framework 3 and introduces two amino acid changes in FR3: H82 to E and N84 to S. The resulting PCR band was cloned into pGem and then the proper size insert was digested with BsiW1 and Bgl II. Cloning of this fragment into HC03 yields HC04.
5. PCR-based mutagenesis using HC04 as template and the following primers, resulted in HC05. Upstream PCR used primers TFHC1s2 and TFHC6 as. Downstream PCR used primers TFHC6s and TFHC1as2. Mutagenic PCR using upstream and downstream PCR products as templates and with primers TFHC1s2 and TFHC1as2 yielded HC05. This step introduced the following amino acid changes in FR3: S76 to T, T77 to S, and F80 to Y.
6. PCR-based mutagenesis using HC05 as template and the following four primers generated HC06. Upstream PCR used primers TFHC2s and TFHC2 as2. Downstream PCR used primers TFHC3s2 and TFHC1as2. Amplification using TFHC2 as2 introduced an amino acid change in FR1: P9 to G. Primer TFHC3s2 changes Q19 to R and T24 to A. PCR using upstream and downstream PCR products as template and with primers TFHC1s2 and TFHC1as2 yielded HC06.
7. A point mutation from I to M in position 2 of FR1 was spontaneously introduced during construction of HC06. PCR amplification using HC06 as template and TFHC1s3 and TFHC1as2 as primers, corrected this erroneous substitution and also introduced an amino acid change in FR1: Q5 to V. The resulting construct was HC07.
8. Construct HC08 was made by PCR-based mutagenesis using HC07 as template and the following primers. TFHC2s and TFHC2 as3 were used for the upstream product. The downstream product was previously amplified using TFHC1s3 and TFHC1as2 (see step 7). The use of primer TFHC2 as3 introduced two amino acid changes in FK1: L11 to V and V12 to K. A spontaneous point mutation resulted in a phenylalanine to leucine (F→L) change at position 64 in CDR2. Further screening and sequencing yielded construct HC08R1, which has the correct sequence of F at position 64 in CDR2.
9. Two constructs, HC11 and HC12, were generated by site-directed mutagenesis from HC07. Two complementary primers TFHC8sP and TFHC8asP were used along with HC07 as template to produce HC11 which contains three amino acid changes in FK1: G9 to P, L11 to V, and V12 to K. Then, HC11 was methylated and column purified for the next round of site directed mutagenesis. PCR using HC11 as a template and the complementary primers TFHC9sL and TFHC0asL generated HC12 which has a mutation from V11 to L in FR1.
10. Construct HC09 was derived from HC12 by performing PCR using HC12 as a template and the complementary primers TFHC10sK and TFHC10asK. HC09 contains an amino acid change: K12 to V in FR1.
11. Construct HC10 was made from HC09. PCR using HC09 as a template and the complementary primers LV-1 and LV-2 resulted in the generation of HC10, which contains a mutation from L11 to V in FR1.

After each mutation step, the partially humanized or fully humanized clones were sequenced and some of these variable domains were later cloned into expression vectors. pJRS 355 or pLAM 356 vector was used to express HC mutants fused to Fc of human IgG1 or IgG4.

FIGS. 3A-D summarize steps 1-11 and shows incremental amino acid changes introduced into FR1-4. Except HC08, all other heavy chain mutants and cH36 contain F at position 64 in CDR2. HC08 has a mutation from F to L at position 64. FIGS. 4B-D show the heavy chain CDR sequences.

### Primers Used for Heavy Chain Humanization

TFHC1s2
5′ TTTCGTACGTCTTGTCCCAGATCCAGCTGCAGCAGTC 3′
TFHC1as2
5′ AGCGAATTCTGAGGAGACTGTGACAGTGGTGCCTTGGCCCCAG 3′
TFHC7s
5′ GTGAGGCAGAGCCCTGGAAAGGGCCTTGAGTGGATTGG 3′
TFHC7as
5′ CCAATCCACTCAAGGCCCTTTCCAGGGCTCTGCCTCAC 3′
TFHC5s
5′ GCATCTCAACAGCCTGAGATCTGAAGACACTGCAGTTTATTTCTG
TG 3′
TFHC5as2
5′ CTGCAGTGTCTTCAGATCTCAGGCTGTTGAGATGCATGAAGGC 3′
TFHC3as
5′ GTCTTCAGATCTCAGGCTGCTGAGCTCCATGAAGGCTGTGGTG 3′
TFHC2s
5′ TACGACTCACTATAGGGCGAATTGG 3′
TFHC6s
5′ CTGTTGACAAGTCTACCAGCACAGCCTACATGGAGCTCAGCAG 3′
TFHC6as
5′ CTGCTGAGCTCCATGTAGGCTGTGCTGGTAGACTTGTCAACAG 3′
TFHC2as2
5′ GCACTGAAGCCCCAGGCTTCACCAGCTCACCTCCAGACTGCTGCA
GC 3′
TFHC3s2
5′ CTGGGGCTTCAGTGCGGGTATCCTGCAAGGCTTCTGGTTACTCAT
TCAC 3′
TFHC1s3
5′ TCGTACGTCTTGTCCCAGATCCAGCTGGTGCAGTCTGGAGGTGAG
C 3′
TFHC2as3
5′ GCACTGAAGCCCCAGGCTTCTTCACCTCACCTCCAGACTGCACC 3′
TFHC9sL
5′ GCAGTCTGGACCTGAGCTGAAGAAGCCTGGGG 3′
TFHC9asL
5′ CCCCAGGCTTCTTCAGCTCAGGTCCAGACTGC 3′
TFHC8sP
5′ GCTGGTGCAGTCTGGACCTGAGGTGAAGAAGCC 3′
TFHC8asP
5′ GGCTTCTTCACCTCAGGTCCAGACTGCACCAGC 3′
TFHC10sK
5′ GCAGTCTGGACCTGAGCTGGTGAAGCCTGGGGCTTC 3′
TFHC10asK
5′ GAAGCCCCAGGCTTCACCAGCTCAGGTCCAGACTGC 3′
LV-1
5′ CAGTCTGGACCTGAGGTGGTGAAGCCTGGG 3′
LV-2
5′ CCCAGGCTTCACCACCTCAGGTCCAGACTG 3′

### D. Generation of Humanized Anti-TF Antibody Light Chain

1. PCR amplification was performed using plasmid pJAIgG4TF.A8 (an expression vector for chimeric H36) as template and primers TFLC1s2.1 and TFLC1 as2. This step introduced a cloning site, AgeI, upstream of the coding region. It also introduced the L1061 mutation in FR4. This step yielded the construct LC03.
2. Site-directed mutagenesis was performed using complementary primers TFLC5s and TFLC5 as and LC03 as template. This step introduced the mutation Q37L in FR2 and added a PstI site for diagnostic purposes. This new construct is named LC04.
3. PCR amplification was performed using LC04 as template and primers TFHC2s and TFLC2 as 1. This step generated Fragment A that will be used in step 6. This step introduced Q11L and L15V mutations in FR1.
4. PCR amplification was performed using LC04 as template and primers TFLC1s2.1 and TFLC1asR. This introduced the KpnI site at the end of LC variable domain. Cloning of this PCR fragment into pGEM yields pGEM04K that will be used in step 6.
5. PCR amplification was performed using LC04 as template and primers TFLC2s and TFLC4 as. This step generated Fragment C that will be used in step 6. Three mutations E17D, S18R in FR1 and A100Q in FR4 were introduced in this step.
6. PCR-based mutagenesis using Fragment A and Fragment C as templates and primers TFHC2s and TFLC4 as yielded Fragment D. Cloning of Fragment D into pGEM04K yielded the construct LC05.
7. PCR amplification was performed using pGEM04K as template and primers TFLC1s2.1 and TFLC4 as. This step generated Fragment H, which is then cloned into pGEM04K. This introduced the A100Q mutation in FR4 and the construct is named LC06.
8. PCR amplification was performed using LC06 as template and primers TFLC1s2.1 and TFLC3 as. This step generated Fragment I that will be used in step 10. This introduced the K70D and the K74T mutations in FR3.
9. PCR amplification was performed using LC06 as template and primers TFLC3s2 and TFLC4 as. This step generated Fragment F that will be used in step 10. This introduced the A80P mutation in FR3.
10. PCR using Fragment I and Fragment F as templates and primers TFLC1s2.1 and TFLC4 as yielded Fragment J. Cloning of Fragment J into pGEM yielded the construct LC07.
11. Site-directed mutagenesis was conduced using complementary primers TFLC08sds and TFLC08sdsa and LC07 as template. This step introduced the mutations V84A and N85T in FR3. This construct is named LC08.
12. The AgeI to EcoO109I fragment from LC05 containing the mutations Q11L, L15V, E17D, S18R and Q37L is cloned into LC08. This yielded the construct LC09.
13. Site-directed mutagenesis was conduced using LC09 as template and the complementary primers LC105 and LC103. This step introduced the T85N mutation in FR3 and yielded the construct LC10.
14. Site-directed mutagenesis was conducted using LC10 as template and the complementary primers LC115 and LC113. This step introduced the D70K mutation in FR3. This yielded the construct LC11.
15. Site-directed mutagenesis was conducted using LC11 as template and the complementary primers LC125a and LC123a. This step introduced the K42Q mutation in FR2. This yielded the construct LC12.

After each mutation step, the partially humanized or fully humanized LC clones were sequenced and some of these variable domains were later cloned into expression vector tKMC180.

### Oligonucleotide Primers Used for Light Chain Humanization

TFLC1as2:
5′ TTCGAAAAGTGTACTTACGTTTGATCTCCAGCTTGGTCCCAG 3′
TFLC1s2.1:
5′ ACCGGTGATATCCAGATGACCCAGTCTCC 3′
TFLC5s:
5′ GGTTAGCATGGTATCTGCAGAAACCAGGG 3′
TFLC5as:
5′ CCCTGGTTTCTGCAGATACCATGCTAACC 3′
TFHC2s:
5′ TACGACTCACTATAGGGCGAATTGG 3′
TFLC2as1:
5′ CCACAGATGCAGACAGGGAGGCAGGAGACTG 3′
TFLC1asR:
5′ TTCGAAAAGTGTACTTACGTTTGATCTCCAGCTTGGTACCAGCACC
GAACG 3′
TFLC2s:
5′ CCTGTCTGCATCTGTGGGAGATAGGGTCACCATCACATGC 3′
TFLC4as:
5′ GATCTCCAGCTTGGTACCCTGACCGAACGTGAATGG 3′
TFLC3as:
5′ GTAGGCTGCTGATCGTGAAAGAAAAGTCTGTGCCAGATCC 3′
TFLC3s2:
5′ CACGATCAGCAGCCTACAGCCTGAAGATTTTGTAAATTATTACTGT
C 3′
TFLC08sds:
5′ GCAGCCTACAGCCTGAAGATTTTGCAACTTATTACTGTCAACAA
G 3′
TFLC08sdsa:
5′ CTTGTTGACAGTAATAAGTTGCAAAATCTTCAGGCTGTAGGCTG
C 3′
LC105:
5′ CAGCAGCCTACAGCCTGAAGATTTTGCAAATTATTACTGTCAAC 3′
LC103:
5′ GTTGACAGTAATAATTTGCAAAATCTTCAGGCTGTAGGCTGCTG 3′
LC115:
5′ CAGTGGATCTGGCACAAAGTTTTCTTTCACGATCAGCAGC 3′
LC113:
5′ GCTGCTGATCGTGAAAGAAAACTTTGTGCCAGATCCACTG 3′
LC125a:
5′ CTGCAGAAACCAGGGCAATCTCCTCAGCTCCTG 3′
LC123a:
5′ CAGGAGCTGAGGAGATTGCCCTGGTTTCTGCAG 3′

FIG. 5A shows the sequence of the human kappa light chain constant domain (SEQ ID NO. ______). FIG. 5B shows the human IgG1 heavy chain constant domain (SEQ ID NO. ______). FIG. 6A shows the hFAT (IgG4) constant domain sequence (SEQ ID NO. ______). FIG. 6B provides the human IgG4 heavy chain constant domain (SEQ ID NO. ______). See also the published U.S. patent application number 20030190705 and references cited therein for additional disclosure relating to the foregoing immunoglobulin constant domain sequences.

### Example 2

### Expression and Purification of Humanized Anti-TF Antibodies

The partially humanized or fully humanized LC and HC clones were cloned into expression vectors. The plasmid tKMC18 was used to express LC mutants fused to human kappa chain, and pJRS 355 or pLAM 356 vector was used to express HC mutants fused to Fc of human IgG1 or IgG4. Some combinations of the HC and LC clones were then co-transfected into COS cells. The transiently expressed IgGs in COS cells were assayed for the whole IgG production and binding to TF by ELISA. For disclosure relating to these particular vectors see the published U.S. patent application number 20030190705 and references cited therein.

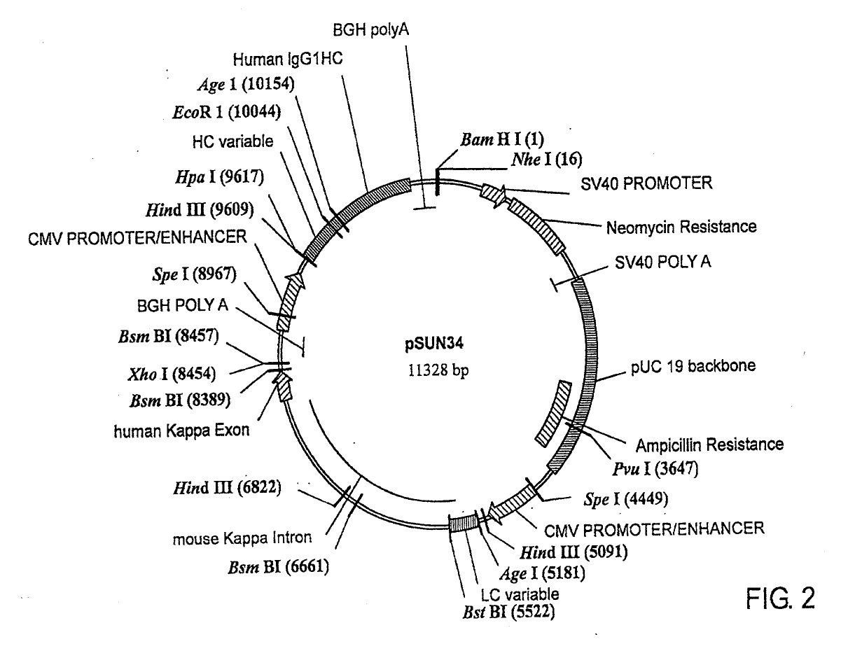

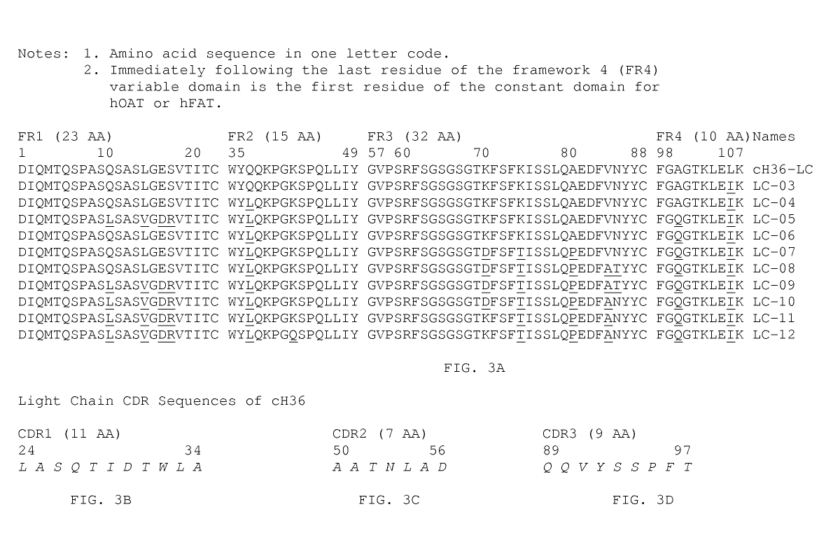

The final fully humanized forms of the anti-TF heavy and light variable domains (combination of HC08 and LC09) were cloned into what is referred to as a Mega expression vector (pSUN34, see FIG. 2) and transfected into CHO and NSO cells for IgG expression. Stably transfected cell lines producing the IgG4κ or IgG1κ humanized anti-TF antibody were cloned. The selected stable cell lines were then used to produce amounts of humanized anti-TF sufficient for analysis. The resulting humanized versions are approximately 100% human in origin (when the CDR sequences are not considered). The humanized IgG4 kappa version (produced by pSUN35) is designated hFAT (humanized IgG Four Anti-Tissue Factor antibody) and the IgG1 kappa version (produced by pSUN34) is designated hOAT (humanized IgG One Anti-Tissue Factor antibody). These fully humanized versions of cH36 are intended for treating chronic indications, such as cancer and inflammatory diseases.

One of the NSO cell lines (OAT-NSO-P10A7) that expresses (combination of HC08 and LC09) was thawed and extended in 10 mL of IMDM medium supplemented with 10% FBS in a 15 mL tube and centrifuged. The cell pellet was resuspended in 10 mL of fresh media and passed to a T25 flask and incubated at 37° C. in 5% CO₂. In order to prepare a sufficient number. of cells to inoculate a hollow fiber bioreactor, the cells were expanded to obtain a total of 6×10⁸ cells. A bioreactor was set up as per manufacturer's instruction manual. The harvested cells were pelleted and resuspended in 60 mL of IMDM containing 35% FBS and injected into the extracapillary space of the bioreactor. Concentrations of glucose and lactate were monitored daily and the harvest material was centrifuged and pooled. The harvested material was tested for anti-TF antibody concentrations by ELISA assay. The pooled sample containing anti-TF antibody (hFAT) were then purified and analyzed as described below.

A. rProtein A Sepharose Fast Flow Chromatography

Recombinant humanized anti-TF monoclonal antibody consists of two light and two heavy chains. Heavy chain is a fusion of mouse variable domain (unaltered or humanized as described above) and human IgG1 or IgG4 Fc domain, while light chain contains mouse variable domain (unaltered or humanized as described above) and human κ domain. It is well established that human IgG Fc region has high affinity for Protein A or recombinant Protein A (rProtein A).

Harvest pools containing humanized anti-TF antibody (hFAT) were adjusted to pH 8.0±0.1 by adding 0.08 ml of 1 M Tris-HCl, pH 8.0 per ml of sample. Then the sample is filtered through low protein-binding 0.22 micron filters (e.g., Nalgene sterile disposable tissue culture filter units with polyethersulfone membrane from Nalge Nunc International, Cat. No. 167-0020). Following sample application, rProtein A column (from Pharmacia) is washed with 5 bed volumes of 20 mM Tris-HCl, pH 8.0 to remove unbound materials such as media proteins. Since the harvest medium contains high content of bovine serum, a stepwise pH gradient wash was used to remove bovine IgG from the column. The stepwise pH gradient was achieved by increasing the relative percentage of Buffer B (100 mM acetic acid) in Buffer A (100 mM sodium acetate). A typical pH stepwise wash employed 20%, 40%, and 60% Buffer B. Elute the column with 100% Buffer B and collect fractions based on A₂₈₀. The pooled fractions were adjusted to pH 8.5 with addition of 1 M Tris base.

### B. Q Sepharose Fast Flow Chromatography

Anion ion exchange chromatography is very effective in separating proteins according to their charges. The eluted and pH-adjusted sample from rProtein A column was diluted with two volumes of water, and the pH is checked and adjusted to 8.5. The sample was then loaded to a 5 ml (1.6×2.5 cm) Q Sepharose Fast Flow equilibrated with 20 mM Tris-HCl, pH 8.5 and the column washed with (1) 5 bed volumes of 20 mM Tris-HCl, pH 8.5; and (2) 4 bed volumes of 20 mM Tris-HCl, pH 8.5 containing 100 mM NaCl. The IgG protein was then eluted with bed volumes of 20 mM Tris-HCl, pH 8.5 containing 500 mM NaCl. The protein peaks were pooled and buffer-exchanged into PBS using ultrafiltration device.

### Example 3

### Septic Shock Model in Rhesus Monkeys

In this model, septic shock was induced by infusion of live E. coli, a gram-negative bacterium (see Taylor et al., J. Clin. Invest. 79:918-825 (1987)) in rhesus monkeys. The shock induced by E. coli causes activation both coagulation and inflammation, ultimately leading to death. The ability of an anti-TF antibody of the present invention to prolong the survival times of rhesus monkeys treated with live E. coli was examined using the rhesus model of septic shock described by Taylor et al., supra. Rhesus monkeys weighing 3-5 kilograms were fasted overnight before study and immobilized the morning of the experiment with ketamine hydrochloride (14 mg/kg, intramuscularly). Sodium pentobarbital was then administered in the cephalic vein through a percutaneous catheter to maintain a light level of surgical anesthesia (2 mg/kg initially and with additional amounts approximately every 20 to 45 minutes for 6 hours). A femoral vein was exposed aseptically and cannulated in one hind limb for sampling blood and administering gentamicin. Gentamicin was administrated by means of 30-minute intravenous infusions. An infusion of 9 mg/kg was administrated at the end of E. coli infusion (t=2 hours). An infusion of 4.5 mg/kg was administrated 6 hours after E. coli infusion. Additional gentamicin (4.5 mg/kg, i.m.) was administrated once daily after day 1 for 3 more days. Each monkey was placed on its side in contact with controlled-temperature heating pads and rectal temperature was monitored. Animals were intubated orally and allowed to breathe spontaneously.

The E. coli strain 086:K61H (ATCC 33985) was freshly prepared in less than 12 hours prior to injection. Each monkey received a 2-hour intravenous infusion of E. coli at a dose of 4×10¹⁰ CFU/kg. Control group monkeys received PBS 30 minutes before infusion of E. coli. Treatment group monkeys received a bolus (2-3 minutes) dose of anti-tissue factor antibody (cH36, diluted in PBS if necessary) 30 minutes before infusion of E. coli (see Timeline for injection schedule). The percutaneous catheter was used to infuse E. coli, PBS and anti-TF antibody.

All monkeys were monitored continuously for 8 hours and observed daily for a maximum of 7 days, for the following: survival time: monitored and recorded hourly; temperature was measured and recorded hourly for the first 8 hours and then once a day for up to 7 days.

Blood samples were collected at the following time points: T=−0.5*, 0**, 1, 2, 4, 6, 24 hours as shown in Table 3 for the analysis of hematological references and inflammatory cytokines. (*T=−0.5, right before injection of cH36 or saline control; **T=0, right before infusion of E coli but 30 min after injection of cH36 or saline control).

TABLE 3
The schedule for collecting blood samples.

| | | Plasma |
|---|---|---|
| Time Point | Hematology | for Analysis |
| Day 1; t = −0.5 hr | X | X |
| (just prior to test article or vehicle | | |
| infusion) | | |
| Day 1; t = 0 hour | X | X |
| (30 minutes after treatment, just | | |
| prior to E. coli infusion) | | |
| Day 1; 1 hour (following E. coli infusion | X | X |
| Day 1; 2 hour (following E. coli infusion) | X | X |
| Day 1; 4 hour (following E. coli infusion) | X | X |
| Day 1; 6 hour (following E. coli infusion) | X | X |
| Day 1; 24 hour (following E. coli infusion) | X | X |
| Volume of whole blood/time point | 1.0 mL | 1.8 mL |
| Anticoagulant | EDTA | Sodium Citrate |

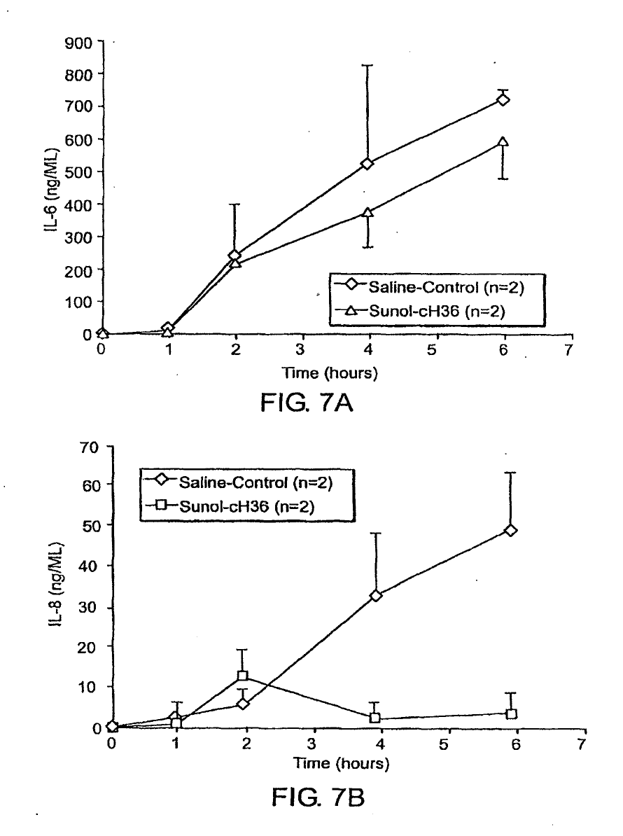

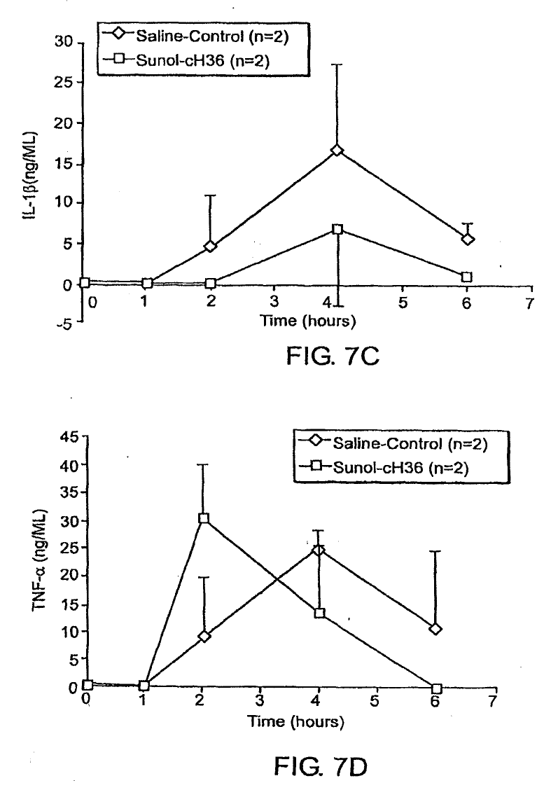

The results of this study are shown in Table 4 and FIG. 7A-D. Anti-TF antibody cH36 protected rhesus monkeys very well from E. coli-induced septic shock when administered as a 10 mg/kg bolus injected intravenously (Table 4), while attenuating inflammatory cytokines IL-8 and IL-1β, and to a lesser extent IL-6 and TNF-α (FIG. 7A-D).

TABLE 4
Protective Effect of cH36 on E coli-induced
Septic Shock in Rhesus Monkeys

| | | | Survival | Average |
|---|---|---|---|---|
| | | | Time | Survival |
| Treatment | Weight (kg) | Sex | (hr) | Time (hr) |
| Saline | 3.6 | F | 8 | |
| | 4.5 | M | 24 | 16 |
| Sunol-cH36 | 3.1 | F | >168 | |
| (10 mg/kg) | 4.0 | M | 54 | >111 |

### Example 4

### Acute Lung Injury Model in Baboons

A. Acute lung injury is an important cause of morbidity and mortality in sepsis. Patients infected with gram-negative sepsis have a high incidence of acute respiratory distress syndrome and multiple organ failure. It has been shown that blocking tissue factor function with active site-inactivated factor VIIa could limit sepsis-induced acute lung injury and other organ damage in baboons (see Welty-Wolf, K. et al., Am. J. Respir. Crit. Care Med. 164:1988 (2001)). A critical pathophysiological feature of the acute respiratory distress syndrome (ARDS) is local activation of extrinsic coagulation and inhibition of fibrinolysis. As the injury evolves, these perturbations cause deposition of fibrin in the microvascular, interstitial and alveolar spaces of the lung leading to capillary obliteration and hyaline membrane formation. Components of the extrinsic coagulation pathway such as TF, thrombin and fibrin signal alterations in inflammatory cell traffic and increases in vascular permeability. Procoagulants and fibrin also promote other key events in the injury including complement activation, production of proinflammatory cytokines, inhibition of fibrinolysis, and remodeling of the injured lung. It has been established that sepsis-induced TF expression activates the extrinsic coagulation cascade in the lung and leads to a procoagulant environment, which results in fibrin deposition and potentiates inflammation. By blockade of the initiating events of extrinsic coagulation, their effects on proinflammatory events in the lungs and disordered fibrin turnover may be corrected and the evolution of severe structural and functional injury may be averted during experimental ARDS. Recent studies demonstrated that preventing initiation of coagulation at TF-Factor VIIa complex with active site inhibited factor VIIa (FVIIai) or TF pathway inhibitor (TFPI) attenuates fibrin deposition and inflammation in sepsis, thereby limiting acute lung injury (ALI) and other organ damage in baboons.

A model for sepsis-induced ALI model has been established in baboons. See Welty-Wolf, K. et al., Am. J. Respir. Crit. Care Med. 164:1988 (2001). In this model, hyperdynamic cardiovascular and systemic inflammatory responses are pre-activated by a priming infusion of killed E. coli. After 12 hours, a second dose of live E. coli is given to the animals to induce pulmonary and renal failure similar to humans with sepsis and ARDS. Using this model, blockade of TF function by FVIIai and TFPI was shown to attenuate systemic inflammatory responses, decrease fibrin deposition in tissues, and prevented lung and renal injury.

In this model, overnight fasted, adult baboons (Papio cyanocephalus) were sedated with intramuscular ketamine (20-25 mg/kg) and intubated. Heavy sedation was maintained with ketamine (3-10 mg/kg/h) and diazepam (0.4-0.8 mg/kg every 2 hours). Animals were mechanically ventilated (21% O₂) with a volume-cycled ventilator and paralyzed intermittently with pancuronium (4 mg intravenously) before respiratory measurements. An indwelling arterial line and a pulmonary artery catheter were placed via femoral cut down for hemodynamic monitoring. All animals received approximately 10⁹ CFU/kg heat-killed E. coli 086:K61H (ATCC 33985) as a 60 min infusion at t=0 h, 12 h before live E. coli. Sepsis was induced at t=12 h by infusing 10¹⁰ CFU/kg of live E. coli in a volume of 50 mL over 60 min. Gentamicin (3 mg/kg i.v.) and Ceftazidime (1 gm i.v.) were administered 60 min after completion of the live E. coli infusion. Fluids were given as needed to maintain pulmonary capillary wedge pressure (PCWP) at 8-12 mmHg and to support blood pressure. Dopamine was used for hypotension when mean arterial pressure (MAP) fell below 65 mmHg despite fluids. After 48 h (36 h after the live bacteria infusion) animals were deeply anesthetized and killed by KCl injection. Physiologic parameters including heart rate (HR), temperature, arterial blood pressure, pulmonary artery pressure, ventilator parameters, and fluid intake were recorded every hour. Measurements were obtained every six hours of cardiac output (CO) by thermodilution, central venous pressure (CVP), PCWP, arterial and mixed venous blood gases, oxygen saturation, oxygen content and hemoglobin (Hgb) as reported. Urinary catheter output was measured every six hours and fluid balance calculated as total i.v. fluid intake minus urine output.

Treatment efficacy for each intervention were assessed by comparing the responses of drug-treated animals with vehicle-treated animals using the physiological, histologic, and biochemical endpoints of lung injury listed below:
Physiological endpoints were evaluated as the alveolar-to-arterial O₂ difference (AaDo₂), pulmonary system compliance (C_{L}), pulmonary artery pressures, pulmonary vascular resistance (PVR) and tissue W/D ratios. Secondary endpoints were fluid volume requirements, serum HCO₃, V_{E} at constant PaCO₂, urine output, creatinine, and systemic DO2, VO₂ and VCO₂.
Pathologic endpoints analyzed were gross tissue appearance and qualitative light microscopic analysis, including fibrin deposition, in lung, kidney, adrenal, and other tissues.
Biochemical endpoints were tissue myeloperoxidase (MPO) levels, total tissue and lavage protein, and lavage LDH. Lung and small bowel edema were measured by wet/dry weights.

Blood samples were drawn at 0, 12, 13, 18, 24, 36, and 48 h. Plasma (from citrated blood) and serum were separated and stored at −80° C. Plasma samples were assayed for interleukin (IL) 6 and IL 8 using ELISA kits (R and D Systems, Inc., Minneapolis, Minn.).

The experimental protocol is summarized in Table 5.

TABLE 5
Experimental Protocol

| Time (hours) | 0 | 6 | 12 | 14 | 18 | 24 | 30 | 36 | 42 | 48 |
|---|---|---|---|---|---|---|---|---|---|---|
| Heat-killed E. coli | X | | | | | | | | | |
| Live E. coli | | | X | | | | | | | |
| Antibiotics | | | | X | | | | | | |
| Vehicle or drug | | | | X | X | X | X | X | X | X |
| Studies* | X | X | X | X | X | X | X | X | X | X |
| Sacrifice | | | | | | | | | | X |

*Studies include serum, plasma, urine and physiology measurements outlined in the detailed methods.

TF blockade was done using a total antibody dose of 3.5 mg/kg for the cH36 Fab and 5.25 mg/kg for cH36. The intravenous loading dose of test article (1.8 mg/kg for cH36 Fab or 2.7 mg/kg for cH36) was begun 2 hours after infusion of live microorganisms, at the time antibiotics were administered, followed by a constant 34-hour infusion of 50 mcg/kg per hour for cH36 Fab or 75 mcg/kg/hour for cH36.

TABLE 6
Experimental Design

| Group | Treatment | # Animals |
|---|---|---|
| 1 | Sepsis + vehicle | 5 |
| 2 | Sepsis + cH36 Fab (3.5 mg/kg total, 1.8 mg/kg | 2 |
| | loading, 50 mcg/kg/hr infusion for 34 hr) | |
| 3 | Sepsis + Sunol-cH36 (5.25 mg/kg total, 2.7 | 5 |
| | mg/kg loading, 75 mcg/kg/hr infusion for 34 hr) | |

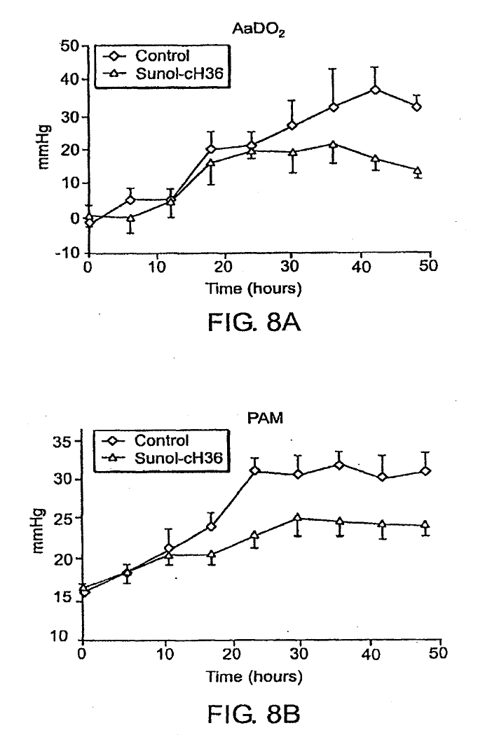

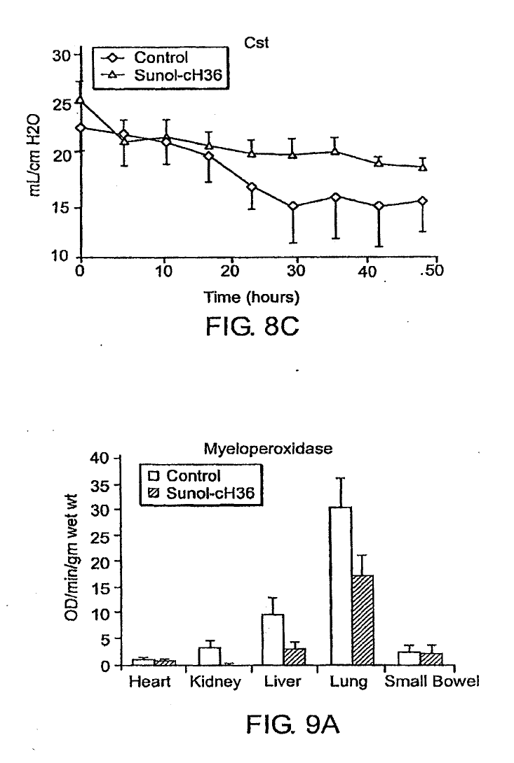

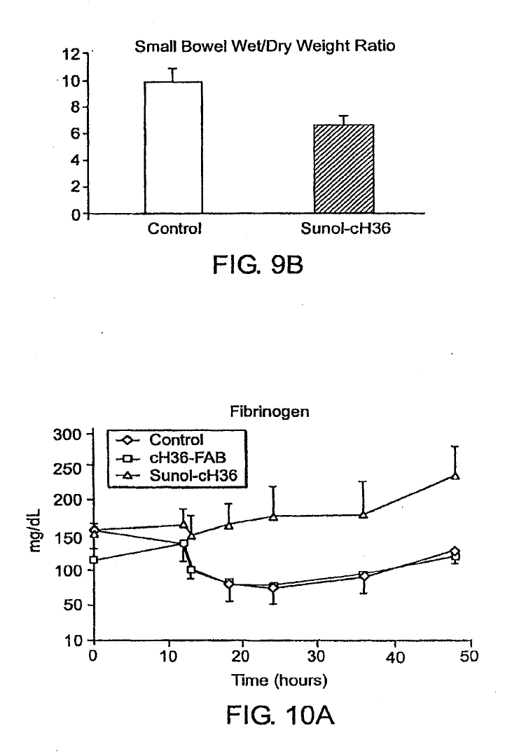

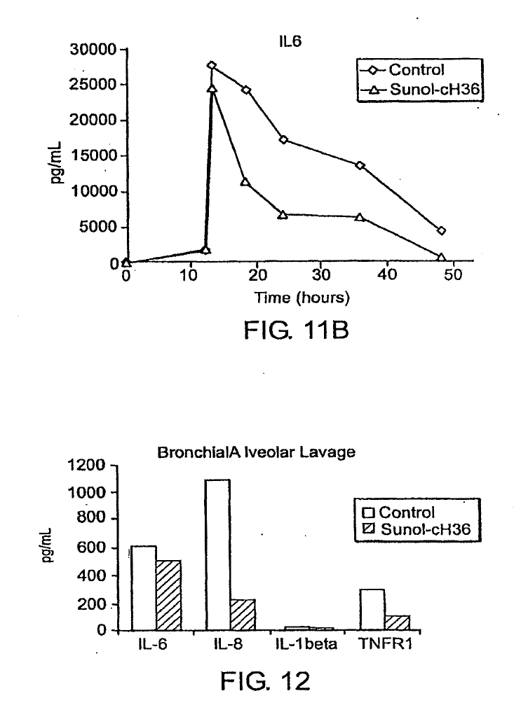

TF blockade with bolus injection of cH36, followed by infusion, attenuated systemic expression of the proinflammatory cytokine IL-8 and IL-6 to a lesser extent (FIGS. 11A-B), and provided partial renal and lung protection in baboons challenged with E. coli. Interim analysis indicates that cH36 administration attenuates increases in mean pulmonary artery pressure, lung system compliance (FIGS. 8A-B) and in the alveolar-arterial oxygen gradient. The kidneys in the animals examined thus far appeared grossly normal at necropsy, the kidney myeloperoxidase levels remained lower (FIG. 9A) and urine output was maintained throughout the experiments. The small bowel wet/dry ratio in the treated animals was significantly lower that in the control animals indicative of less edema in the cH36 treated animals (FIG. 9B).

These data indicate that coagulation blockade targeting TF with Sunol-cH36 prevents inflammation and the development of organ injury in gram-negative sepsis. These data also show, for the first time, a new strategy for management of ARDS in humans with anticoagulant.

B. The following data further show that cH36 and cH36-Fab are useful in preventing lung injury in septic baboons.

Briefly, the objective of this part of the example is to further confirm effects of Sunol-cH36 and cH36-Fab on procoagulant-fibrinolytic balance and inflammation in the lung and relate them to the structural and gas exchange abnormalities in ALI in an experimental sepsis model in baboons. See section A, above.

All baboons (Papio cyanocephalus) were mechanically ventilated (21% O₂), anesthetized, and given a dose of heat-killed E. coli (1×10⁹ CFU/kg) intravenously 12 hours prior to the onset of live E. coli sepsis (1-2×10¹⁰ CFU/kg). The study design consisted of three groups of baboons as shown in Table 7, below. The baboons in the first group (n=6) were administered vehicle (PBS) and served as controls. The animals in the second group (n=3) were injected with cH36-Fab (3.5 mg/kg total, 1.8 mg/kg bolus loading dose followed by constant infusion of 50 mcg/kg/hr for 34 hours). The third group (n=6) received cH36 (5.25 mg/kg total, 2.7 mg/kg bolus loading dose followed by a constant infusion of 75 mcg/kg/hr for 34 hours). The intravenous loading dose of drug (1.8 mg/kg for cH36-Fab or 2.7 mg/kg for cH36) was started 2 hours after infusion of live microorganisms (at the 14-hour time point) followed by a constant infusion of 50 mcg/kg per hour for cH36-Fab or 75 mcg/kg/hour for cH36 until the end of the experiment at 48 hours. Antibiotics were administered at the 14-hour time point. Treatment efficacy was assessed by comparison of the responses of the treated animals with the controls using physiological, histological and biochemical parameters of lung injury.

TABLE 7
Experimental Design

| Group | Treatment | No. of Baboons |
|---|---|---|
| 1 | Sepsis + vehicle | 6 |
| 2 | Sepsis + cH36-Fab (3.5 mg/kg total, 1.8 mg/kg | 3 |
| | loading, 50 mcg/kg/hr infusion for 34 hr) | |
| 3 | Sepsis + cH36 (5.25 mg/kg total, 2.7 mg/kg | 6 |
| | loading, 75 mcg/kg/hr infusion for 34 hr) | |

From the foregoing data, it can be concluded that animals treated with cH36 had a less hyperdynamic systemic response to sepsis.

The following Materials and Methods were used as needed to conduct the experiments in this Example. They were also used elsewhere in this disclosure as indicated.

C. Materials and Methods

Mechanically ventilated (21% O₂), anesthetized baboons (Papio cyanocephalus) were given a dose of heat-killed E. coli (1×10⁹ CFU/kg) intravenously 12 hours prior to the onset of live E. coli sepsis (1-2×10¹⁰ CFU/kg). The intravenous loading dose of drug (1.8 mg/kg for cH36-Fab or 2.7 mg/kg for cH36) was started 2 hours after infusion of live microorganisms (14 hours), at the time antibiotics were administered, followed by a constant infusion of 50 mcg/kg per hour for cH36-Fab or 75 mcg/kg/hour for cH36. The total antibody dose was 3.5 mg/kg for the cH36-Fab and 5.25 mg/kg for cH36. Treatment was initiated after the onset of gram-negative sepsis because we have previously shown that TF blockade is effective as a rescue strategy. Because initial studies suggested greater efficacy and no harmful effects of whole antibody compared to Fab, comparisons were made between untreated sepsis controls and septic animals treated with cH36 whole antibody. Experimental groups are as shown in Table 7. Statistical analyses used ANOVA for physiologic data and t test or Mann Whitney U for biochemical and BAL data. Data are expressed as mean±sem and p values are shown.

Animals were handled in accordance with appropriate guidelines. The animals were divided randomly into treatment and sepsis control groups. After an overnight fast, each animal was sedated with intramuscular ketamine (20-25 mg/kg) and intubated. Heavy sedation was maintained with ketamine (3-10 mg/kg/h) and diazepam (0.4-0.8 mg/kg every 2 hours). Animals were ventilated with a volume-cycled ventilator and paralyzed intermittently with pancuronium (4 mg intravenously) before respiratory measurements. The FiO₂ was 0.21, tidal volume 12 ml/kg, positive end-expiratory pressure 2.5 cm H₂O, and a rate adjusted to maintain an arterial PCO₂ of 40 mmHg. An indwelling arterial line and a pulmonary artery catheter were placed via femoral cut down for hemodynamic monitoring. The animals received gentamicin sulfate (3 mg/kg iv) and ceftazidime (1 gm iv) two hours after the start of live E. coli infusion (14 hours). The animals were supported with intravenous volume infusion (Ringer's lactate) at a rate sufficient to maintain the pulmonary capillary wedge pressure (PCWP) at 8 to 12 mmHg. Dopamine was used if needed to maintain mean arterial pressure of 60 mmHg. After 48 h (36 h after the live bacteria infusion) animals were deeply anesthetized and killed by KCl injection. Predefined early termination criteria included refractory hypotension (MAP less than 60 mmHg despite dopamine and adequate PCWP), hypoxemia (need for FIO₂ greater than 40%), or refractory metabolic acidosis (pH<7.10 with normal PaCO₂). The experimental protocol was the same as that shown in Table 5, above.

Physiological parameters including heart rate (HR), temperature, arterial blood pressure, pulmonary artery pressure, ventilator parameters, and fluid intake were recorded every hour. Measurements were obtained every six hours of cardiac output (CO) by thermodilution, central venous pressure (CVP), PCWP, arterial and mixed venous blood gases, oxygen saturation, oxygen content and hemoglobin (Hgb). Urinary catheter output was measured every six hours and fluid balance calculated as total iv. fluid intake minus urine output.

Blood samples were drawn at 0, 12, 13, 18, 24, 36, and 48 h. Complete blood counts were performed on whole blood (Sysmex-1000 Hemocytometer; Sysmex, Inc., Long Grove, Ill.). Plasma (from citrated blood) and serum were separated and stored at −80° C. Fibrinogen was measured using an ST4 mechanical coagulation analyzer (Diagnostics Stago, Parsippany, N.J.). Prothrombin time (PT) and activated partial thromboplastin time (aPTT) were measured in duplicate, and antithrombin III (ATIII) activity was measured on an MDA coagulation analyzer (Organon Teknika, Durham, N.C.) with a chromogenic assay and expressed as % of the kit standard. ELISA was used to measure plasma thrombin-antithrombin (TAT) complexes (Dade Behring, Deerfield, Ill.) in plasma and BAL. cH36 and cH36-Fab levels in blood and BAL were measured by Sunol Molecular (Miramar, Fla.). Serum samples were assayed for interleukin 1β (IL-1β), IL-6, IL-8, and TNF receptor-1 (TNFR-1) using ELISA kits (R and D Systems, Inc., Minneapolis, Minn.). Blood creatinine was measured with standard clinical techniques.

Tissues were collected at autopsy as follows: After the experiments the thorax was opened, the left mainstem bronchus ligated, and the left lung removed. BAL was performed on the left upper lobe with 240 mL 0.9% saline. Samples of lung tissue from the left lower lobe were manually inflated and immersed in 4% paraformaldehyde for light microscopy. Four samples were taken at random from the remainder of the left lung for wet/dry weight determination taking care to avoid large vascular and bronchial structures. Additional samples from lung, kidney, liver, small bowel, heart, and adrenal were flash frozen in liquid nitrogen and stored at −80° C. The entire right lung was inflation-fixed for 15 min at 30 cm fixative pressure with 2% glutaraldehyde in 0.85 M Na cacodylate buffer (pH 7.4). Additional tissue from kidney, liver, small bowel, heart, and adrenal was fixed by immersion in 4% paraformaldehye. Four samples of small bowel were selected randomly for wet/dry weight determination.

Myeloperoxidase (MPO) activity and protein concentration of lung homogenates and protein and lactate dehydrogenase (LDH) concentrations of BAL fluid were measured as described (Am J Resp Crit Care Med 1998; 157:938). MPO activity was expressed as a change in absorbance/min/g wet weight tissue. LDH values were expressed in units of activity per liter (U/L).

An important objective of the following Examples was to determine the effects of cH36 and cH36-Fab on procoagulant-fibrinolytic balance and inflammation in the lung. It was also an objective to correlate these parameters to the structural and gas exchange abnormalities in ALI in an experimental sepsis model in baboons.

D. Results: Treatment with cH36 Prevents Fibrinogen Depletion in Baboons with E. coli Sepsis.

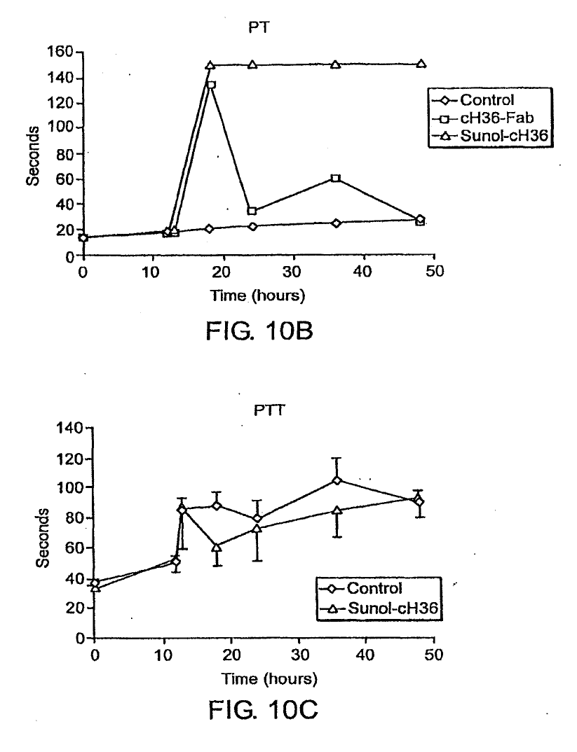

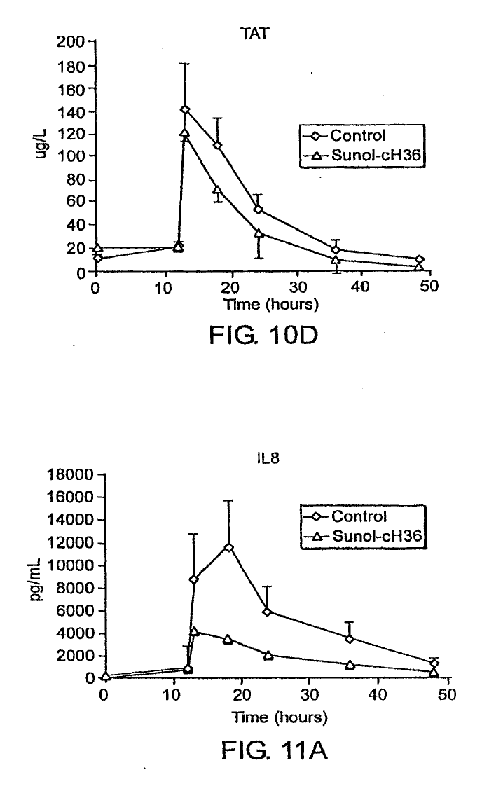

Briefly, cH36 treatment attenuated fibrinogen depletion and TAT complex formation, consistent with inhibition of TF-dependent activation of coagulation. In sepsis controls, fibrinogen decreased to approximately 50% of initial values, but in animals treated with cH36 mean fibrinogen levels did not drop below baseline values (FIG. 10A, p<0.01 versus sepsis controls). PT increased in sepsis controls during sepsis due to a progressive coagulopathy, and also increased in treated animals due to pharmacologic effect of the drug infusion. PT and fibrinogen values in the three cH36-Fab treated animals are also shown for comparison. The decreased values in the cH36-Fab compared to whole antibody treated animals suggest that the Fab fragment has a lower affinity for TF (FIG. 10A,B). PTT increased in both groups and was not significantly different. TAT complexes increased after infusion of live bacteria in both groups, peaking at 14 h and then decreasing. Peak TAT value was lower and levels decreased more rapidly in animals treated with cH36 (FIG. 10D, p<0.01). The difference in TAT complex formation was not due to differences in ATIII levels, as ATIII decreased similarly in the two groups, to 35-40% of initial values by the end of the experiment.

E. Results: Treatment with Sunol-cH36 Attenuates Sepsis-Induced Acute Lung Injury

cH36 decreased ALI in baboons with established E. coli sepsis, attenuating sepsis-induced abnormalities in gas exchange, pulmonary hypertension, and loss of pulmonary system compliance. These physiologic data are shown in FIGS. 8A-C. Alveolar arterial oxygen gradient (AaDO₂, mmHg) increased in both groups after infusion of killed bacteria and progressively worsened in the sepsis control group after the onset of live bacterial sepsis at t=12 h. Compared to septic control animals, treatment with cH36 prevented progressive deterioration in gas exchange during sepsis (p=0.001, FIG. 8A). One animal in the sepsis control group required supplemental oxygen after the onset of live bacterial sepsis for progressive hypoxemia. One animal in the cH36 treated group also required oxygen, but only from 18-22 hours, during which oxygenation gradually improved and supplemental O₂ was weaned back to room air. At the end of the experiment the AaDO₂ in that animal had recovered to initial values measured prior to infusion of heat-killed or live bacteria. Sepsis-induced increase in mean pulmonary artery pressure (PAM, mmHg) was attenuated by cH36 (p<0.0001 vs. untreated sepsis controls, FIG. 8B) but there was no difference in pulmonary vascular resistance, suggesting that this was due to effect on cardiac output in the treated animals. cH36 also prevented the decrease in pulmonary system compliance (Cst in mL/cm H₂O) seen in sepsis control animals (p<0.01, FIG. 8C). The PaCO₂ was controlled within the normal range, between 35-45 mmHg in both groups, but was slightly higher in sepsis controls (p=0.03) despite 20% higher minute ventilation (V_{E}, 1/min, p=0.015), suggesting cH36 attenuated sepsis-induced increases in dead space (Table 7).

At post-mortem, the lungs from sepsis control animals were dense and hemorrhagic. The gross appearance of the lungs from animals treated with cH36 was improved and in some animals appeared the same as lungs from normal uninjured baboons. Lung wet/dry weights were not significantly different in the two groups, 6.32±0.66 in septic controls compared to 5.57±0.34 in cH36 treated animals (p=NS, normal reference range is 4.6-5.0). BAL protein and LDH were also not significantly different between the two groups. BAL protein was 1.0±0.3 in septic controls compared to 1.0±0.4 in cH36 treated animals, and LDH was 23.9±10.6 in septic controls compared to 10.6±3 in cH36 treated animals. Neutrophil accumulation, as measured by myeloperoxidase (MPO) activity (OD/min/gm wet wt), was decreased over 40% in cH36 treated animals (p=0.07).

Lung histology showed protection in septic animals treated with cH36. The lungs of sepsis control animals had thickened alveolar septae, patchy alveolar edema and hemorrhage, and infra-alveolar inflammatory cell infiltration with macrophages and PMNs. Lungs of treated animals had improved alveolar septal architecture, decreased alveolar PMN infiltration, less alveolar edema, and no alveolar hemorrhage.

### Example 5

### Treatment with cH36 Improves Renal Function and Reduces Organ Injury in Sepsis

Materials and methods used to perform the present Example have been described previously. See eg., Example 4.

Treatment with cH36 improved renal function in sepsis. Urine output was significantly higher after infusion of live E. coli in animals treated with cH36 compared to untreated controls (p<0.001). This was not due to differences in resuscitation because fluid balance and systemic hemodynamics were similar in the two groups. Blood pH and serum [HCO3⁻] were lower in untreated animals (both p<0.0001), and values were consistent with mixed metabolic and respiratory acidosis in sepsis controls. Serum creatinine was not different in the two groups at the end of the experiments, and there was variability in the treated group due to one animal that was not protected.

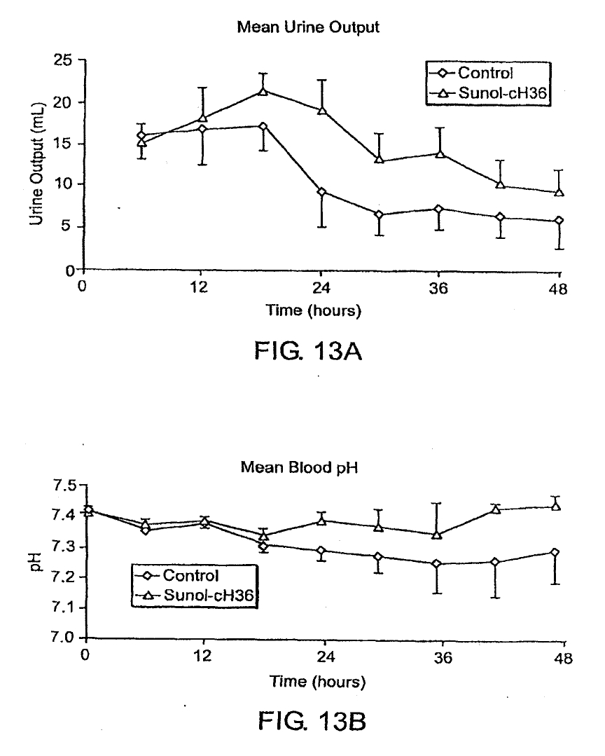

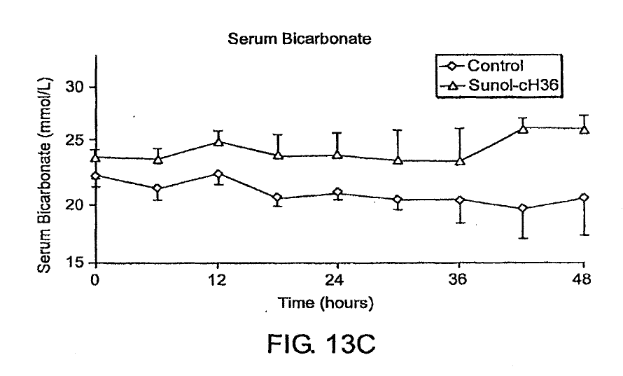

See FIGS. 13A-C (showing mean urine output (13A), mean blood pH (13B), and serum bicarbonate levels (13C) with control and cH36 antibodies.

Kidneys from untreated animals were swollen and hemorrhagic at post mortem but appeared more normal in cH36 treated animals. H&E stained sections of the kidneys of untreated animals had patchy to extensive areas of acute tubular necrosis (ATN) and glomerular damage. The kidneys of treated animals, except for a few small foci of ATN, showed normal renal architecture. MPO values were significantly decreased in kidneys from treated animals (p=0.01).

Other organ injury was also improved in the cH36 treated animals. Adrenals from untreated animals were swollen and hemorrhagic and small bowel was grossly edematous. In contrast, adrenals and small bowel appeared almost normal in animals treated with cH36. Small bowel wet/dry weights were decreased in septic animals treated with cH36 (6.46±0.62 in Sunol-cH36 treated vs. 9.70±1.05 in untreated sepsis control animals, p=0.01). Histology confirmed improvement in small bowel edema. Adrenals were also protected from sepsis, with decrease in congestion and hemorrhage, and areas of cellular damage were markedly diminished. In addition to the decreased neutrophil content in the lung and kidney, cH36 treatment significantly decreased PMN content in the liver (p=0.05), and histology showed decreased injury to hepatocytes.

### Example 6

### cH36 Treatment Attenuated Sepsis-Induced Anemia

Materials and methods used to perform the present Example have been explained above. See eg., Example 4.

Both groups of animals developed neutropenia, thrombocytopenia and anemia after infusion of live E. coli (see Table 8, below). Hemoglobin (Hgb) decreased in both groups but was significantly higher in septic animals treated with cH36 (8.1±0.7 versus 10.8±0.8 g/dL at 48 hours, p<0.0001). Although platelets declined more rapidly in untreated animals (p<0.0001), the clinical significance of this difference is not clear. All animals developed progressive thrombocytopenia after the infusion of live E. coli and mean platelet counts were approximately 30,000 or less in both groups at the end of the experiment. WBC reached a nadir of approximately 1,000-1,500 (×10³/μL) in both groups two hours after the infusion (t=14 h) and progressively increased to baseline levels by the end of the experiment (12,680±2,012 in treated vs. 10,500±1,336 in untreated animals, p=0.07). Two sepsis control animals developed self-limited hematuria. Most animals in both groups had some blood tinged secretions associated with suctioning at some point in the study, but there was no clinical evidence of significant hemorrhage (i.e. hematuria, hemoptysis, or bleeding from intravenous or arterial catheter sites) in the cH36 treated animals and no severe or life-threatening bleeding complications occurred in either group.

TABLE 8

| Time (h) | | 0 | 12 | 18 | 24 | 36 | 48 | p value |
|---|---|---|---|---|---|---|---|---|
| Hg/dL) | Sepsis | 11.2 ± 0.3 | 10.6 ± 0.3 | 10.4 ± 0.5 | 10.4 ± 0.8 | 8.7 ± 0.8 | 8.1 ± 0.7 | <0.0001 |
| | cH36 | 12.1 ± 0.5 | 11.8 ± 0.4 | 12.2 ± 0.5 | 12.4 ± 0.6 | 10.9 ± 0.8 | 10.8 ± 0.8 | |
| | cH36 Fab | 11.7 ± 0.3 | 11.5 ± 0.5 | 12.5 ± 0.8 | 11.4 ± 0.3 | 11.3 ± 0.4 | 9.6 ± 0.1 | |
| Platelets | Sepsis | 194 ± 15 | 126 ± 12 | 59 ± 7 | 33 ± 5 | 24 ± 5 | 26 ± 9 | <0.0001 |
| | cH36 | 206 ± 15 | 167 ± 16 | 110 ± 13 | 64 ± 12 | 40 ± 4 | 30 ± 4 | |
| | cH36 Fab | 197 ± 8 | 146 ± 4 | 87 ± 10 | 31 ± 5 | 21 ± 4 | 25 | |
| HR | Sepsis | 95 ± 6 | 116 ± 10 | 125 ± 9 | 129 ± 9 | 125 ± 5 | 127 ± 16 | <0.01 |
| (beats/min) | cH36 | 84 ± 3 | 101 ± 4 | 135 ± 13 | 123 ± 11 | 105 ± 12 | 99 ± 9 | |
| | cH36 Fab | 75 ± 7 | 89 ± 9 | 107 ± 14 | 109 ± 9 | 98 ± 15 | 98 ± 9 | |
| MAP | Sepsis | 111 ± 2 | 104 ± 2 | 99 ± 9 | 102 ± 7 | 79 ± 10 | 78 ± 13 | NS |
| (mmHg) | cH36 | 102 ± 3 | 93 ± 3 | 97 ± 5 | 92 ± 6 | 78 ± 9 | 86 ± 5 | |
| | cH36 Fab | 108 ± 4 | 85 ± 4 | 89 ± 9 | 78 ± 8 | 89 ± 4 | 63 ± 4 | |
| CO/kg | Sepsis | 0.16 ± 0.01 | 0.20 ± 0.02 | 0.23 ± 0.04 | 0.22 ± 0.03 | 0.22 ± 0.04 | 0.23 ± 0.02 | <0.0001 |
| | cH36 | 0.15 ± 0.01 | 0.18 ± 0.01 | 0.16 ± 0.02 | 0.16 ± 0.02 | 0.15 ± 0.02 | 0.18 ± 0.01 | |
| | cH36 Fab | 0.12 ± 0.01 | 0.14 ± 0.01 | 0.12 ± 0.01 | 0.15 ± 0.01 | 0.14 ± 0.01 | 0.14 ± 0.02 | |
| D_{O2}/kg | Sepsis | 23.2 ± 2.2 | 28.1 ± 3.1 | 28.6 ± 4.1 | 26.2 ± 2.0 | 22.7 ± 24.3 | 20.1 ± 1.2 | NS |
| | cH36 | 23.8 ± 1.8 | 28.0 ± 1.0 | 23.8 ± 2.4 | 24.9 ± 3.5 | 23.1 ± 2.6 | 24.6 ± 2.1 | |
| | cH36 Fab | 18.1 ± 1.6 | 21.8 ± 2.1 | 19.5 ± 1.8 | 20.9 ± 2.7 | 21.1 ± 1.8 | 16.4 ± 1.4 | |
| V_{O2}/kg | Sepsis | 5.4 ± 0.8 | 6.3 ± 1.4 | 6.7 ± 0.6 | 6.8 ± 1.3 | 5.7 ± 0.6 | 5.6 ± 0.7 | NS |
| | cH36 | 5.6 ± 0.4 | 6.6 ± 0.2 | 6.2 ± 0.4 | 5.6 ± 0.7 | 6.1 ± 0.6 | 6.2 ± 0.3 | |
| | cH36 Fab | 4.0 ± 0.3 | 4.2 ± 0.2 | 3.7 ± 0.4 | 4.6 ± 0.3 | 3.7 ± 0.2 | 4.2 ± 0.2 | |
| SVR*kg | Sepsis | 56602 ± 5459 | 41539 ± 5049 | 35290 ± 4941 | 37788 ± 5675 | 30713 ± 9152 | 26161 ± 5750 | *** |
| | cH36 | 50833 ± 3782 | 37936 ± 895 | 49953 ± 8998 | 46811 ± 6290 | 37432 ± 3612 | 36058 ± 4362 | |
| | cH36 Fab | 69095 ± 2475 | 43890 ± 5700 | 52763 ± 7023 | 39545 ± 7126 | 46570 ± 5101 | 32243 ± 8576 | |
| PCWP | Sepsis | 10 ± 1 | 11 ± 1 | 10 ± 1 | 11 ± 1 | 12 ± 1 | 11 ± 1 | NS |
| | cH36 | 10 ± 0 | 10 ± 0 | 9 ± 1 | 9 ± 1 | 11 ± 2 | 9 ± 1 | |
| | cH36 Fab | 12 ± 0 | 14 ± 1 | 10 ± 1 | 10 ± 1 | 11 ± 1 | 12 ± 2 | |
| V_{E} | Sepsis | 3.5 ± 0.3 | 3.5 ± 0.2 | 3.8 ± 0.2 | 4.2 ± 0.2 | 5.0 ± 0.5 | 5.1 ± 0.4 | =0.015 |
| | cH36 | 3.7 ± 0.1 | 3.9 ± 0.2 | 4.0 ± 0.2 | 4.3 ± 0.2 | 4.4 ± 0.3 | 4.3 ± 0.2 | |
| | cH36 Fab | 4.3 ± 0.2 | 4.4 ± 0.2 | 4.5 ± 0.1 | 5.0 ± 04 | 6.4 ± 0.8 | 6.2 ± 0.9 | |
| | | | 15 hr | | | | | |
| cH36 | cH36 | 0 | 49.3 ± 5.5 | 51.8 ± 3.3 | 60.2 ± 6.0 | 53.0 ± 5.6 | 58.0 ± 8.3 | |
| (ng/mL) | cH36 Fab | 0 | ± | ± | ± | ± | ± | |

Clinical and histological evaluations are performed to confirm that anti-tissue factor injections inhibited the development of rheumatoid arthritis in these mice.

Referring to the foregoing Examples and discussion, it was found that animals treated with cH36 had a less hyperdynamic systemic response to sepsis. Most systemic hemodynamic measurements, including mean arterial pressure (MAP), PCWP, systemic vascular resistance*kg (SVR*kg), VO₂, and DO₂, were not altered by treatment with cH36 (Table 8). Hypotension responded to IV fluids and dopamine infusion in both groups. Nine of the 12 animals survived until the scheduled termination point of the protocol. Overall, septic animals treated with cH36 were less hyperdynamic, without further increases in cardiac output (CO/kg) after the onset of live bacterial sepsis, and had less tachycardia and higher systemic vascular resistance*kg (SVR*kg) at the end of the experiment (Table 7, for instance). In some instances, treated animals needed dopamine and fluid support as did the untreated controls and MAP and SVR were not detectably different between the two groups. Survival is not intended for use as an endpoint as all animals were sacrificed at the 48 hour time point. Three septic animals were treated with cH36-Fab to assess for differences between whole antibody and Fab fragment. As expected, the whole antibody was often more effective then the Fab fragment.

### Example 7

### Impact of cH36 on Pro-Inflammatory Cytokine Levels

Cytokine levels were measured in serum and BAL fluid along lines already described above (eg., Example 5). In the circulation, cH36 treatment attenuated IL-8 (p<0.01, FIG. 12) but had no detectable effect on IL-1β, or TNFR-1. In BAL fluid, cH36 attenuated elevations in IL-6, IL-8 and TNFR-1. BAL cytokine levels are shown in Table 9 below. We also measured soluble thrombomodulin (sTM) and found no differences in treated vs. untreated animals in serum or BAL. The data show that inhibiting coagulation using Sunol-cH36 to block the FX binding site on TF-FVIIa complex attenuates acute lung injury at least in part through effects on proinflammatory cytokine levels in the alveolar compartment. This could be an effect on local cytokine production and/or leakage of proteins from the circulation across the alveolar epithelium.

TABLE 9
BAL cytokine levels

| | Sepsis control | Sunol-cH36 | p value | cH36-Fab |
|---|---|---|---|---|
| IL-6 (pg/mL) | 600 ± 269 | 506 ± 522 | 0.05 | 1159 ± 435 |
| IL-8 (pg/mL) | 1081 ± 448 | 224 ± 176 | 0.037 | 1478 ± 755 |
| IL-1β (pg/mL) | 6.9 ± 1.5 | 7.9 ± 3.1 | NS | 8.5 ± 1.8 |
| TNFR1 (pg/mL) | 294 ± 126 | 107 ± 31 | 0.09 | 167 ± 93 |
| sTM (ng/mL) | 1.67 ± 0.54 | 0.9 ± 0.11 | NS | 2.17 ± 0.72 |

Certain results from Examples 4-7 can be summarized as follows:

Most systemic hemodynamic measurements, including mean arterial pressure (MAP), oxygen consumption (VO₂/kg), and oxygen delivery (DO₂/kg), were not altered by treatment with cH36 (Table 8). Mean pulmonary capillary wedge pressure (PCWP) was slightly higher in sepsis control animals (p<0.01) but both groups were within the set parameters of the study. Systemic vascular resistance*kg (SVR*kg) was slightly higher in treated animals (p<0.05, Table 3). Hypotension responded to IV fluids and dopamine infusion in both groups. Nine of the 12 animals survived until the scheduled termination point of the protocol. Two sepsis control animals died prior to scheduled termination from ALI, with refractory hypoxemia and respiratory acidosis, one at 30 hours and one at 38 hours. One animal in the H36 group was not protected and died at 36 hours of refractory hypotension and metabolic acidosis. In that animal, lack of protection correlated with lower drug levels. Overall, septic animals treated with cH36 were less hyperdynamic, without further increases in cardiac output (CO/kg) after the onset of live bacterial sepsis, and had less tachycardia and higher systemic vascular resistance per kg (SVR*kg) at the end of the experiment (Table 7).

### Example 8

### Treatment of Baboons with cH36-Fab (Fab Fragment)

Three septic animals were treated with cH36-Fab look for any differences between use of whole antibody and Fab fragment. Treatment protocols generally followed procedures already discussed above. Although the group was too small for detailed statistical analyses, the data suggested that the Fab fragment had effect in attenuating sepsis-induced activation of coagulation, with increased TAT values and depletion of fibrinogen similar to septic controls. However, that effect was less pronounced then results achieved with whole antibody. Correspondingly, there was less consistent improvement in gas exchange (AaDO2), pulmonary hypertension (PAM), and lung compliance (Cst) than in animals treated with whole cH36 antibody. Biochemically, lung MPO values were similar to septic controls. Without wishing to be bound to theory, the difference in effect between cH36 and its Fab fragment may be due to the lower affinity of the Fab fragment for TF in the animal model.

### Example 9

### Anti-Tissue Factor Inhibition of Collagen-Induced Arthritis in Transgenic Mice Expressing Human Tissue Factor

Collagen-induced arthritis (CIA) is an established experimental model of rheumatoid arthritis which is induced in susceptible strains of mice following immunization with type II collagen. In addition to the immune mediated mechanism in the pathogenesis of rheumatoid arthritis, tissue factor initiated activation of the coagulation cascade has also been implicated in the progression of the disease. Fibrin deposition in the affected joints resulting from the activation of coagulation is believed to contribute to synovial thickening and joint inflammation. To determine whether treatment with anti-tissue factor antibodies will prevent development of rheumatoid arthritis mice will be injected with cH36.

To induce CIA 7-12 week old mice are injected intradermally at the base of the tail with 100 μg of type II collagen emulsified in Complete Freund's Adjuvant and given a boost injection with 100 μg of type II collagen emulsified in Incomplete Freund's Adjuvant at day 21. Immediately prior to the boost injection mice are given 0.3 mg anti-tissue factor antibody by IV injection (the control group are injected with PBS). Anti-tissue factor antibody injections (0.3 mg) are subsequently given weekly for the duration of the study (the control group are injected with PBS).

Animals are assessed for redness and swelling of the limbs and a clinical score is allocated three times per week. The clinical severity is scored as follows: 1 point for each swollen digit except the thumb (maximum 4), 1 Point for the tarsal or carpal joint, and one point for the metatarsal or metacarpal joint with a maximum score of 6 for a hindpaw and 5 for a forepaw. Each paw is graded individually, the cumulative clinical arthritic score per mouse can reach a maximum of 22 points.

The present provisional application has information relating to published U.S. patent application No. 20030190705 which application is related to U.S. application Ser. No. 09/990,586 as filed on Nov. 21, 2001, which application claims priority to U.S. Provisional Application U.S. Ser. No. 60/343,306 as filed on Oct. 29, 2001. The U.S. application Ser. No. 09/990,586 is related to U.S. application Ser. No. 09/293,854 (now U.S. Pat. No. 6,555,319) which application is a divisional of U.S. application Ser. No. 08/814,806 (now U.S. Pat. No. 5,986,065) and the U.S. application Ser. No. 10/230,880 claims priority to U.S. application Ser. No. 09/990,586. The disclosures of the U.S. application Ser. Nos. 10/230,880, 09/990,586, 60/343,306 and U.S. Pat. Nos. 5,986,065 and 6,555,319 are each incorporated by reference. Also incorporated by reference is the disclosure of published U.S. patent application No. 20030190705.

The present provisional application is further related to U.S. provisional application Ser. No. 60/480,254 as filed on Jun. 19, 2003, the disclosure of which is incorporated by reference.

The invention has been described in detail with reference to preferred embodiments thereof. However, it will be appreciated that those skilled in the art, upon consideration of the disclosure, may make modification and improvements within the spirit and scope of the invention. All references disclosed herein are incorporated herein by reference.

1-12. (canceled)

13. A method for treating acute lung injury (ALI) or acute respiratory distress syndrome (ARDS) in a mammal comprising administering to the mammal a therapeutically effective amount of at least one humanized antibody, chimeric antibody, or fragment thereof that comprises complementarity determining region (CDR) of light chain and heavy chain having the following amino acid sequence:
Light chain CDR 1: SEQ ID NO. 126;
Light chain CDR 2: SEQ ID NO. 127;
Light chain CDR 3: SEQ ID NO. 128;
Heavy chain CDR 1: SEQ ID NO. 185;
Heavy chain CDR 2: SEQ ID NO. 186;
Heavy chain CDR 3: SEQ ID NO. 187, and
wherein the administration is sufficient to prevent or treat the condition in the mammal.

14. A method for treating acute lung injury (ALI) or acute respiratory distress syndrome (ARDS) in a mammal comprising administering to the mammal a therapeutically effective amount of at least one humanized antibody, chimeric antibody, or fragment thereof that comprises complementarity determining region (CDR) of light chain and heavy chain having the following amino acid sequence:
Light chain CDR 1: SEQ ID NO. 126;
Light chain CDR 2: SEQ ID NO. 127;
Light chain CDR 3: SEQ ID NO. 128;
Heavy chain CDR 1: SEQ ID NO. 188;
Heavy chain CDR 2: SEQ ID NO. 189;
Heavy chain CDR 3: SEQ ID NO. 190, and
wherein the administration is sufficient to prevent or treat the condition in the mammal.

15. A method for treating acute lung injury (ALI) or acute respiratory distress syndrome (ARDS) in a mammal comprising administering to the mammal a therapeutically effective amount of a chimeric antibody or fragment thereof that comprises a light chain variable domain comprising the amino acid sequence of SEQ ID NO: 2 and a heavy chain variable domain comprising the amino acid sequence of SEQ ID NO: 4, and wherein the administration is sufficient to prevent or treat the condition in the mammal.

16. The method according to any one of claims 13 and 14, wherein the light chain of the humanized antibody or fragment thereof further comprises:
(a) a first framework (FR1) which comprises a sequence having at least 95% homology to any one of SEQ ID NO: 82 or 94;
(b) a second framework (FR2) which comprises a sequence having at least 95% homology to any one of SEQ ID NO: 83, 91, or 123,
(c) a third framework (FR3) which comprises a sequence having at least 95% homology to any one of SEQ ID NO: 84, 104, 108, 116, or 120; and
(d) a fourth framework (FR4) which comprises a sequence having at least 95% homology to any one of SEQ ID NO: 85, 89, or 97 and
wherein the heavy chain of the humanized antibody or fragment thereof further comprises:
(a) a first framework (FR1) which comprises a sequence having at least 95% homology to any one of SEQ ID NO: 129, 133, 153, 157, 161, 169, 173, 177, or 181;
(b) a second framework (FR2) which comprises a sequence having at least 95% homology to any one of SEQ ID NO: 130 or 138;
(c) a third framework (FR3) which comprises a sequence having at least 95% homology to any one of SEQ ID NO: 131, 143, 147, or 151; and
(d) a fourth framework (FR4) which comprises a sequence having at least 95% homology to any one of SEQ ID NO: 132 or 136.

17. The method according to any one of claims 13 and 14, wherein the light chain of the humanized antibody or fragment thereof further comprises:
(a) a first framework (FR1) which comprises a sequence of SEQ ID NO: 82 or 94;
(b) a second framework (FR2) which comprises a sequence of SEQ ID NO: 83, 91, or 123,
(c) a third framework (FR3) which comprises a sequence of SEQ ID NO: 84, 104, 108, 116, or 120; and
(d) a fourth framework (FR4) which comprises a sequence of SEQ ID NO: 85, 89, or 97; and
wherein the heavy chain of the humanized antibody or fragment thereof further comprises:
(a) a first framework (FR1) which comprises a sequence of SEQ ID NO: 129, 133, 153, 157, 161, 169, 173, 177, or 181;
(b) a second framework (FR2) which comprises a sequence of SEQ ID NO: 130 or 138;
(c) a third framework (FR3) which comprises a sequence of SEQ ID NO: 131, 143, 147, or 151; and
(d) a fourth framework (FR4) which comprises a sequence of SEQ ID NO: 132 or 136.

18. The method according to claim 16, wherein the first framework (FR1) of the light chain variable region of the antibody comprises one or more of the following amino acid changes as compared to SEQ ID NO: 82: Q11 to L; L15 to V; E17 to D; and S18 to R.

19. The method according to claim 16, wherein the second framework (FR2) of the light chain variable region of the antibody comprises one or more of the following amino acid change as compared to SEQ ID NO: 83: Q3 to L and K8 to Q.

20. The method according to claim 16, wherein the third framework (FR3) of the light chain variable region of the antibody comprises one or more of the following amino acid changes as compared to SEQ ID NO: 84: K14 to D, K18 to T, A24 to P, V28 to A, and N29 to T.

21. The method according to claim 16, wherein the fourth framework (FR4) of the light chain variable region of the antibody comprises one or more of the following amino acid changes as compared to SEQ ID NO: 85: A3 to Q; and L9 to I.

22. The method according to claim 16, wherein the first framework (FR1) of the heavy chain variable region of the antibody comprises one or more of the following amino acid changes as compared to SEQ ID NO: 129, E1 to Q; Q5 to V; P9 to G; L11 to V; V12 to K; Q19 to R; and T24 to A.

23. The method according to claim 16, wherein the second framework (FR2) of the heavy chain variable region of the antibody comprises one or more of the following amino acid changes as compared to SEQ ID NO: 130, H6 to P; and S9 to G.

24. The method according to claim 16, wherein the third framework (FR3) of the heavy chain variable region of the antibody comprises one or more of the following amino acid changes as compared to SEQ ID NO: 131: S10 to T; T11 to S; F14 to Y; H16 to E; N18 to S; T21 to R; D23 to E; and S25 to T.

25. The method according to claim 16, wherein the fourth framework (FR4) of the heavy chain variable region of the antibody comprises the following amino acid change as compared to SEQ ID NO: 132: L7 to V.

26. The method according to any one of claims 13 and 14, wherein the antibody further comprises light chain FR1, FR2, FR3 and FR4 comprising SEQ ID NOs: 110 to 113, respectively, and heavy chain FR1, FR2, FR3 and FR4 comprising SEQ ID NOs:161 to 164, respectively.

27. The method according to any one of claims 13 and 14, wherein the antibody further comprises a constant region for the light chain of SEQ ID NO: 191 and a constant region for the heavy chain of SEQ ID NO: 192.

28. The method according to any one of claims 13 and 14, wherein the antibody further comprises a constant region for the light chain of SEQ ID NO: 193 and a constant region for the heavy chain of SEQ ID NO: 194.

29. The method according to claim 15, wherein the antibody further comprises a constant region for the light chain of SEQ ID NO: 191 and a constant region for the heavy chain of SEQ ID NO: 192.

30. The method according to claim 15, wherein the antibody further comprises a constant region for the light chain of SEQ ID NO: 193 and a constant region for the heavy chain of SEQ ID NO: 194.

31. The method according to claim 26, wherein the antibody further comprises a constant region for the light chain of SEQ ID NO: 191 and a constant region for the heavy chain of SEQ ID NO: 192.

32. The method according to claim 26, wherein the antibody further comprises a constant region for the light chain of SEQ ID NO: 193 and a constant region for the heavy chain of SEQ ID NO: 194.

33. The method according to any one of claims 13, 14, and 15, wherein the antibody or fragment exhibits at least one property selected from the group consisting of: (1) a dissociation constant (K_{d}) for TF of less than about 0.5 nM; and (2) an affinity constant (K_{A}) for TF of at least about 3×10⁹ M⁻¹.

34. The method according to any one of claims 13, 14, and 15, wherein the antibody or fragment has a binding specificity for TF that is equal to or greater than that of the antibody that is obtained from cell line H36.D2.B7 as deposited with the ATCC under accession no. HB-12255.

35. The method according to any one of claims 13, 14, and 15, wherein the antibody or fragment is a humanized antibody that has an IgG1 or IgG4 isotype.

36. The method according to any one of claims 13, 14, and 15, wherein the antibody or fragment is an Fab, Fab′, or F(ab′)₂ fragment.

37. The method according to any one of claims 13, 14, and 15, wherein the antibody or fragment is a single-chain immunoglobulin.

38. The method according to any one of claims 13, 14, and 15, wherein the antibody is a monoclonal antibody.

39. The method according to any one of claims 13, 14, and 15, wherein the mammal to be treated is a primate.

40. The method according to claim 39, wherein the primate is a human.

41. The method according to any one of claims 13, 14, and 15, wherein the treatment attenuates IL-6, IL-8, IL-1β, TNF-α, or TNFR levels in the mammal after at least five hours.

42. The method according to any one of claims 13, 14, and 15, wherein the amount of the antibody or fragment to be administered to the mammal is sufficient to inhibit platelet deposition by at least 50%.

43. The method according to any one of claims 13, 14, and 15, wherein the amount of the antibody or fragment to be administered to the mammal is between 0.01 and 25 mg/kg.
